SEQUENCE LISTING (133 sequences)

SEQ ID NO: 1 - protein, 118 aa, Artificial Sequence
  Description of Artificial Sequence Synthetic polypeptide
EVQLVESGGGLVQPGGSLRLSCAASGFTFXXXPMXWVRQAPGKGLEWVAXIXXSDXXXXYRDSVKGRFTISRDNAKXSLYLQMNSLRAEDTAVYYCARGYYNSPFAYWGQGTLVTVSS

SEQ ID NO: 2 - protein, 118 aa, Artificial Sequence
  Description of Artificial Sequence Synthetic polypeptide
EVQLVESGGGLVQPGGSLRLSCAASGFTFSHFPMAWVRQAPGKGLEWVASISSSDSTTNYRDSVKGRFTISRDNAKNSLYLQMNSLRAEDTAVYYCARGYYNSPFAYWGQGTLVTVSS

SEQ ID NO: 3 - protein, 6 aa, Artificial Sequence
  Description of Artificial Sequence Synthetic peptide
SHFPMA

SEQ ID NO: 4 - protein, 17 aa, Artificial Sequence
  Description of Artificial Sequence Synthetic peptide
SISSSDSTTNYRDSVKG

SEQ ID NO: 5 - protein, 9 aa, Artificial Sequence
  Description of Artificial Sequence Synthetic peptide
GYYNSPFAY

SEQ ID NO: 6 - protein, 118 aa, Artificial Sequence
  Description of Artificial Sequence Synthetic polypeptide
EVQLVESGGGLVQPGGSLRLSCAASGFTFKNFPMAWVRQAPGKGLEWVATISSSDLSTNYRDSVKGRFTISRDNAKNSLYLQMNSLRAEDTAVYYCARGYYNSPFAYWGQGTLVTVSS

SEQ ID NO: 7 - protein, 6 aa, Artificial Sequence
  Description of Artificial Sequence Synthetic peptide
KNFPMA

SEQ ID NO: 8 - protein, 17 aa, Artificial Sequence
  Description of Artificial Sequence Synthetic peptide
TISSSDLSTNYRDSVKG

SEQ ID NO: 9 - protein, 118 aa, Artificial Sequence
  Description of Artificial Sequence Synthetic polypeptide
EVQLVESGGGLVQPGGSLRLSCAASGFTFRNFPMAWVRQAPGKGLEWVASISSSDGTTNYRDSVKGRFTISRDNAKSSLYLQMNSLRAEDTAVYYCARGYYNSPFAYWGQGTLVTVSS

SEQ ID NO: 10 - protein, 6 aa, Artificial Sequence
  Description of Artificial Sequence Synthetic peptide
RNFPMA

SEQ ID NO: 11 - protein, 17 aa, Artificial Sequence
  Description of Artificial Sequence Synthetic peptide
SISSSDGTTNYRDSVKG

SEQ ID NO: 12 - protein, 118 aa, Artificial Sequence
  Description of Artificial Sequence Synthetic polypeptide
EVQLVESGGGLVQPGGSLRLSCAASGFTFRHFPMTWVRQAPGKGLEWVASISSSDGTINYRDSVKGRFTISRDNAKNSLYLQMNSLRAEDTAVYYCARGYYNSPFAYWGQGTLVTVSS

SEQ ID NO: 13 - protein, 6 aa, Artificial Sequence
  Description of Artificial Sequence Synthetic peptide
RHFPMT

SEQ ID NO: 14 - protein, 17 aa, Artificial Sequence
  Description of Artificial Sequence Synthetic peptide
SISSSDGTINYRDSVKG

SEQ ID NO: 15 - protein, 118 aa, Artificial Sequence
  Description of Artificial Sequence Synthetic polypeptide
EVQLVESGGGLVQPGGSLRLSCAASGFTFRNFPMAWVRQAPGKGLEWVATISSSDGTPAYRDSVKGRFTISRDNAKNSLYLQMNSLRAEDTAVYYCARGYYNSPFAYWGQGTLVTVSS

SEQ ID NO: 16 - protein, 17 aa, Artificial Sequence
  Description of Artificial Sequence Synthetic peptide
TISSSDGTPAYRDSVKG

SEQ ID NO: 17 - protein, 118 aa, Artificial Sequence
  Description of Artificial Sequence Synthetic polypeptide
EVQLVESGGGLVQPGGSLRLSCAASGFTFRYFPMAWVRQAPGKGLEWVAAISGSDGTASYRDSVKGRFTISRDNAKNSLYLQMNSLRAEDTAVYYCARGYYNSPFAYWGQGTLVTVSS

SEQ ID NO: 18 - protein, 6 aa, Artificial Sequence
  Description of Artificial Sequence Synthetic peptide
RYFPMA

SEQ ID NO: 19 - protein, 17 aa, Artificial Sequence
  Description of Artificial Sequence Synthetic peptide
AISGSDGTASYRDSVKG

SEQ ID NO: 20 - protein, 118 aa, Artificial Sequence
  Description of Artificial Sequence Synthetic polypeptide
EVQLVESGGGLVQPGGSLRLSCAASGFTFNHFPMAWVRQAPGKGLEWVATISSSDWTPYYRDSVKGRFTISRDNAKNSLYLQMNSLRAEDTAVYYCARGYYNSPFAYWGQGTLVTVSS

SEQ ID NO: 21 - protein, 6 aa, Artificial Sequence
  Description of Artificial Sequence Synthetic peptide
NHFPMA

SEQ ID NO: 22 - protein, 17 aa, Artificial Sequence
  Description of Artificial Sequence Synthetic peptide
TISSSDWTPYYRDSVKG

SEQ ID NO: 23 - protein, 118 aa, Artificial Sequence
  Description of Artificial Sequence Synthetic polypeptide
EVQLVESGGGLVQPGGSLRLSCAASGFTFQKYPMAWVRQAPGKGLEWVATISCSDGITHYRDSVKGRFTISRDNAKNSLYLQMNSLRAEDTAVYYCARGYYNSPFAYWGQGTLVTVSS

SEQ ID NO: 24 - protein, 6 aa, Artificial Sequence
  Description of Artificial Sequence Synthetic peptide
QKYPMA

SEQ ID NO: 25 - protein, 17 aa, Artificial Sequence
  Description of Artificial Sequence Synthetic peptide
TISCSDGITHYRDSVKG

SEQ ID NO: 26 - protein, 118 aa, Artificial Sequence
  Description of Artificial Sequence Synthetic polypeptide
EVQLVESGGGLVQPGGSLRLSCAASGFTFRHFPMAWVRQAPGKGLEWVATISSSDGATYYRDSVKGRFTISRDNAKSSLYLQMNSLRAEDTAVYYCARGYYNSPFAYWGQGTLVTVSS

SEQ ID NO: 27 - protein, 6 aa, Artificial Sequence
  Description of Artificial Sequence Synthetic peptide
RHFPMA

SEQ ID NO: 28 - protein, 17 aa, Artificial Sequence
  Description of Artificial Sequence Synthetic peptide
TISSSDGATYYRDSVKG

SEQ ID NO: 29 - protein, 118 aa, Artificial Sequence
  Description of Artificial Sequence Synthetic polypeptide
EVQLVESGGGLVQPGGSLRLSCAASGFTFRHFPMAWVRQAPGKGLEWVASISSSDGTSNYRDSVKGRFTISRDNAKSSLYLQMNSLRAEDTAVYYCARGYYNSPFAYWGQGTLVTVSS

SEQ ID NO: 30 - protein, 17 aa, Artificial Sequence
  Description of Artificial Sequence Synthetic peptide
SISSSDGTSNYRDSVKG

SEQ ID NO: 31 - protein, 118 aa, Artificial Sequence
  Description of Artificial Sequence Synthetic polypeptide
EVQLVESGGGLVQPGGSLRLSCAASGFTFGHFPMAWVRQAPGKGLEWVATISSSDGATNYRDSVKGRFTISRDNAKNSLYLQMNSLRAEDTAVYYCARGYYNSPFAYWGQGTLVTVSS

SEQ ID NO: 32 - protein, 6 aa, Artificial Sequence
  Description of Artificial Sequence Synthetic peptide
GHFPMA

SEQ ID NO: 33 - protein, 17 aa, Artificial Sequence
  Description of Artificial Sequence Synthetic peptide
TISSSDGATNYRDSVKG

SEQ ID NO: 34 - protein, 13 aa, Artificial Sequence
  Description of Artificial Sequence Synthetic peptide
ASTKGPSVFPLAP

SEQ ID NO: 35 - protein, 12 aa, Artificial Sequence
  Description of Artificial Sequence Synthetic peptide
TVAAPSVFIFPP

SEQ ID NO: 36 - protein, 12 aa, Artificial Sequence
  Description of Artificial Sequence Synthetic peptide
ASTKGPSVFPLA

SEQ ID NO: 37 - protein, 11 aa, Artificial Sequence
  Description of Artificial Sequence Synthetic peptide
TVAAPSVFIFP

SEQ ID NO: 38 - protein, 11 aa, Artificial Sequence
  Description of Artificial Sequence Synthetic peptide
ASTKGPSVFPL

SEQ ID NO: 39 - protein, 10 aa, Artificial Sequence
  Description of Artificial Sequence Synthetic peptide
TVAAPSVFIF

SEQ ID NO: 40 - protein, 10 aa, Artificial Sequence
  Description of Artificial Sequence Synthetic peptide
ASTKGPSVFP

SEQ ID NO: 41 - protein, 9 aa, Artificial Sequence
  Description of Artificial Sequence Synthetic peptide
TVAAPSVFI

SEQ ID NO: 42 - protein, 9 aa, Artificial Sequence
  Description of Artificial Sequence Synthetic peptide
ASTKGPSVF

SEQ ID NO: 43 - protein, 8 aa, Artificial Sequence
  Description of Artificial Sequence Synthetic peptide
TVAAPSVF

SEQ ID NO: 44 - protein, 8 aa, Artificial Sequence
  Description of Artificial Sequence Synthetic peptide
ASTKGPSV

SEQ ID NO: 45 - protein, 7 aa, Artificial Sequence
  Description of Artificial Sequence Synthetic peptide
TVAAPSV

SEQ ID NO: 46 - protein, 7 aa, Artificial Sequence
  Description of Artificial Sequence Synthetic peptide
ASTKGPS

SEQ ID NO: 47 - protein, 6 aa, Artificial Sequence
  Description of Artificial Sequence Synthetic peptide
TVAAPS

SEQ ID NO: 48 - protein, 6 aa, Artificial Sequence
  Description of Artificial Sequence Synthetic peptide
ASTKGP

SEQ ID NO: 49 - protein, 5 aa, Artificial Sequence
  Description of Artificial Sequence Synthetic peptide
TVAAP

SEQ ID NO: 50 - protein, 14 aa, Artificial Sequence
  Description of Artificial Sequence Synthetic peptide
GGGGSGGGGSGGGG

SEQ ID NO: 51 - protein, 13 aa, Artificial Sequence
  Description of Artificial Sequence Synthetic peptide
GGSGGGGSGGGGS

SEQ ID NO: 52 - protein, 13 aa, Artificial Sequence
  Description of Artificial Sequence Synthetic peptide
GGGGSGGGGSGGG

SEQ ID NO: 53 - protein, 12 aa, Artificial Sequence
  Description of Artificial Sequence Synthetic peptide
GGSGGGGSGGGG

SEQ ID NO: 54 - protein, 12 aa, Artificial Sequence
  Description of Artificial Sequence Synthetic peptide
GGGGSGGGGSGG

SEQ ID NO: 55 - protein, 11 aa, Artificial Sequence
  Description of Artificial Sequence Synthetic peptide
GGSGGGGSGGG

SEQ ID NO: 56 - protein, 11 aa, Artificial Sequence
  Description of Artificial Sequence Synthetic peptide
GGGGSGGGGSG

SEQ ID NO: 57 - protein, 10 aa, Artificial Sequence
  Description of Artificial Sequence Synthetic peptide
GGSGGGGSGG

SEQ ID NO: 58 - protein, 10 aa, Artificial Sequence
  Description of Artificial Sequence Synthetic peptide
GGGGSGGGGS

SEQ ID NO: 59 - protein, 9 aa, Artificial Sequence
  Description of Artificial Sequence Synthetic peptide
GGSGGGGSG

SEQ ID NO: 60 - protein, 9 aa, Artificial Sequence
  Description of Artificial Sequence Synthetic peptide
GGGGSGGGG

SEQ ID NO: 61 - protein, 8 aa, Artificial Sequence
  Description of Artificial Sequence Synthetic peptide
GGSGGGGS

SEQ ID NO: 62 - protein, 8 aa, Artificial Sequence
  Description of Artificial Sequence Synthetic peptide
GGGGSGGG

SEQ ID NO: 63 - protein, 7 aa, Artificial Sequence
  Description of Artificial Sequence Synthetic peptide
GGSGGGG

SEQ ID NO: 64 - protein, 7 aa, Artificial Sequence
  Description of Artificial Sequence Synthetic peptide
GGGGSGG

SEQ ID NO: 65 - protein, 6 aa, Artificial Sequence
  Description of Artificial Sequence Synthetic peptide
GGSGGG

SEQ ID NO: 66 - protein, 6 aa, Artificial Sequence
  Description of Artificial Sequence Synthetic peptide
GGGGSG

SEQ ID NO: 67 - protein, 5 aa, Artificial Sequence
  Description of Artificial Sequence Synthetic peptide
GGSGG

SEQ ID NO: 68 - protein, 14 aa, Artificial Sequence
  Description of Artificial Sequence Synthetic peptide
TPAPLPAPLPAPTT

SEQ ID NO: 69 - protein, 13 aa, Artificial Sequence
  Description of Artificial Sequence Synthetic peptide
TPAPLPAPLPAPT

SEQ ID NO: 70 - protein, 12 aa, Artificial Sequence
  Description of Artificial Sequence Synthetic peptide
TPAPLPAPAPTT

SEQ ID NO: 71 - protein, 11 aa, Artificial Sequence
  Description of Artificial Sequence Synthetic peptide
TPAPLPAPAPT

SEQ ID NO: 72 - protein, 10 aa, Artificial Sequence
  Description of Artificial Sequence Synthetic peptide
TPAPLPAPTT

SEQ ID NO: 73 - protein, 9 aa, Artificial Sequence
  Description of Artificial Sequence Synthetic peptide
TPAPLPAPT

SEQ ID NO: 74 - protein, 8 aa, Artificial Sequence
  Description of Artificial Sequence Synthetic peptide
TPAPLPTT

SEQ ID NO: 75 - protein, 7 aa, Artificial Sequence
  Description of Artificial Sequence Synthetic peptide
TPAPLPT

SEQ ID NO: 76 - protein, 6 aa, Artificial Sequence
  Description of Artificial Sequence Synthetic peptide
TPAPTT

SEQ ID NO: 77 - protein, 5 aa, Artificial Sequence
  Description of Artificial Sequence Synthetic peptide
TPAPT

SEQ ID NO: 78 - protein, 122 aa, Artificial Sequence
  Description of Artificial Sequence Synthetic polypeptide
EVQLQESGPGLVKPSETLSLTCTVSGFSLSDYGVSWIRQPPGKGLEWIGLIWGSGDTYYNSPLKSRLTISKDNSKSQVSLKLSSVTAADTAVYYCAKQTNIWAYDLYSMDYWGQGTLVTVSS

SEQ ID NO: 79 - protein, 122 aa, Artificial Sequence
  Description of Artificial Sequence Synthetic polypeptide
EVQLQESGPGLVKPSETLSLTCTVSGFSLSEFGVSWIRQPPGKGLEWIGLIWGGGDTYYNSPLKSRLTISKDNSKSQVSLKLSSVTAADTAVYYCAKQRNLWAYDLYGMDYWGQGTLVTVSS

SEQ ID NO: 80 - protein, 122 aa, Artificial Sequence
  Description of Artificial Sequence Synthetic polypeptide
EVQLQESGPGLVKPSETLSLTCTVSGFSLSDYGVSWIRQPPGKGLEWIGLIWGSGDTYYNSPLKSRLTISKDTSKSQVSLKLSSVTAADTAVYYCAKQTNLWAYDLYSMDYWGQGTLVTVSS

SEQ ID NO: 81 - protein, 122 aa, Artificial Sequence
  Description of Artificial Sequence Synthetic polypeptide
EVQLQESGPGLVKPSETLSLTCTVSGFSLRDYGVSWIRQPPGKGLEWLGLIWGSGDTYYNSPLKSRLTISKDTSKSQVSLKLSSVTAADTAVYYCAKQTNIWGYDLYGMDYWGQGTLVTVSS

SEQ ID NO: 82 - protein, 122 aa, Artificial Sequence
  Description of Artificial Sequence Synthetic polypeptide
EVQLQESGPGLVKPSETLSLTCTVSGFSLSDYGVSWIRQPPGKGLEWIGLIWGGGDTYYNSPLKSRLTISKDNSKSQVSLKLSSVTAADTAVYYCARQTNLWAYDLYSMDYWGQGTLVTVSS

SEQ ID NO: 83 - protein, 119 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSS

SEQ ID NO: 84 - protein, 119 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSS

SEQ ID NO: 85 - protein, 121 aa, Artificial Sequence
  Description of Artificial Sequence Synthetic polypeptide
EVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 86 - protein, 126 aa, Artificial Sequence
  Description of Artificial Sequence Synthetic polypeptide
EVQLVQSGAEVKKPGSSVKVSCKASGGSFGGYGIGWVRQAPGQGLEWMGGITPFFGFADYAQKFQGRVTITADESTTTAYMELSGLTSDDTAVYYCARDPNEFWGGYYSTHDFDSWGQGTTVTVSS

SEQ ID NO: 87 - protein, 126 aa, Artificial Sequence
  Description of Artificial Sequence Synthetic polypeptide
EVQLVQSGAEVKKPGESVKISCKASGGSFRSYGISWVRQAPGQGLEWMGGITHFFGITDYAQKFQGRVTITADESTTTAYMELSGLTSDDTAVYYCAREPNDFWGGYYDTHDFDSWGQGTTVTVSS

SEQ ID NO: 88 - protein, 108 aa, Artificial Sequence
  Description of Artificial Sequence Synthetic polypeptide
DIQMTQSPSSLSASVGDRVTITCQTSTDIDDDLNWYQQKPGKAPKLLISLASTLRPGVPSRFSGSGSGTDFTFTISSLQPEDFATYYCLQSDRLPLTFGQGTKLEIKR

SEQ ID NO: 89 - protein, 108 aa, Artificial Sequence
  Description of Artificial Sequence Synthetic polypeptide
DIQMTQSPSSLSASVGDRVTITCQTSQDIDMDLNWYQQKPGKAPKLLISQGSTLWPGVPSRFSGSGSGTDFTFTISSLQPEDFATYYCLQTDSFPLTFGQGTKLEIKR

SEQ ID NO: 90 - protein, 108 aa, Artificial Sequence
  Description of Artificial Sequence Synthetic polypeptide
DIQMTQSPSSLSASVGDRVTITCQASQDIDDDLNWYQQKPGKAPKLLISLASILRPGVPSRFSGSGSGTDFTFTISSLQPEDFATYYCLQSDSFPLTFGQGTKLEIKR

SEQ ID NO: 91 - protein, 108 aa, Artificial Sequence
  Description of Artificial Sequence Synthetic polypeptide
DIQMTQSPSSLSASVGDRVTITCQASQDIDMDLNWYQQKPGKAPKLLISQANTLPPGVPSRFSGSGSGTDFTFTISSLQPEDFATYYCLQSDWLPLTFGQGTKLEIKR

SEQ ID NO: 92 - protein, 108 aa, Artificial Sequence
  Description of Artificial Sequence Synthetic polypeptide
DIQMTQSPSSLSASVGDRVTITCQASTDIDDDLNWYQQKPGKAPKLLISLGSTLRPGVPSRFSGSGSGTDFTFTISSLQPEDFATYYCLQSDRLPLTFGQGTKLEIKR

SEQ ID NO: 93 - protein, 108 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKR

SEQ ID NO: 94 - protein, 108 aa, Artificial Sequence
  Description of Artificial Sequence Synthetic polypeptide
DIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 95 - protein, 108 aa, Artificial Sequence
  Description of Artificial Sequence Synthetic polypeptide
EIVLTQSPDFQSVTPKEKVTITCRASQDIGSELHWYQQKPDQPPKLLIKYASHSTSGVPSRFSGSGSGTDFTLTINGLEAEDAGTYYCHQTDSLPYTFGPGTKVDIKR

SEQ ID NO: 96 - protein, 108 aa, Artificial Sequence
  Description of Artificial Sequence Synthetic polypeptide
EIVLTQSPDFQSVTPKEKVTITCRASQNIGSELHWYQQKPDQSPKLLIKYASHSISGVPSRFSGSGSGTDFTLTINGLEAEDAATYYCHQSDTLPHTFGQGTKVDIKR

SEQ ID NO: 97 - protein, 250 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPSVFPEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 98 - protein, 253 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPSVFPLAPEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 99 - protein, 252 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPSVFPLAEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 100 - protein, 246 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 101 - protein, 247 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPSEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 102 - protein, 248 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPSVEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 103 - protein, 250 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPSVFPEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 104 - protein, 250 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSGGGGSGGGGSEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 105 - protein, 253 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPSVFPLAPEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 106 - protein, 246 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 107 - protein, 248 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPSVEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 108 - protein, 251 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPSVFPLEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 109 - protein, 249 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPSVFEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 110 - protein, 256 aa, Artificial Sequence
  Description of Artificial Sequence Synthetic polypeptide
EVQLQESGPGLVKPSETLSLTCTVSGFSLSDYGVSWIRQPPGKGLEWLGLIWGSGDTYYNSPLKSRLTISKDTSKSQVSLKLSSVTAADTAVYYCAKQTNIWGYDLYGMDYWGQGTLVTVSSASTKGPSVFPLAPEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 111 - protein, 256 aa, Artificial Sequence
  Description of Artificial Sequence Synthetic polypeptide
EVQLQESGPGLVKPSETLSLTCTVSGFSLSDYGVSWIRQPPGKGLEWLGLIWGSGDTYYNSPLKSRLTISKDTSKSQVSLKLSSVTAADTAVYYCAKQTNIWGYDLYGMDYWGQGTLVTVSSGGGGSGGGGSGGGEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 112 - protein, 255 aa, Artificial Sequence
  Description of Artificial Sequence Synthetic polypeptide
EVQLQESGPGLVKPSETLSLTCTVSGFSLRDYGVSWIRQPPGKGLEWLGLIWGSGDTYYNSPLKSRLTISKDTSKSQVSLKLSSVTAADTAVYYCAKQTNIWGYDLYGMDYWGQGTLVTVSSASTKGPSVFPLAEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 113 - protein, 256 aa, Artificial Sequence
  Description of Artificial Sequence Synthetic polypeptide
EVQLQESGPGLVKPSETLSLTCTVSGFSLRDYGVSWIRQPPGKGLEWLGLIWGSGDTYYNSPLKSRLTISKDTSKSQVSLKLSSVTAADTAVYYCAKQTNIWGYDLYGMDYWGQGTLVTVSSASTKGPSVFPLAPEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 114 - protein, 251 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSGGGGSGGGGSGEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 115 - protein, 254 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSGGGGSGGGGSGGGGEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 116 - protein, 247 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSGGGGSGGEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 117 - protein, 248 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSGGGGSGGGEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 118 - protein, 249 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSGGGGSGGGGEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 119 - protein, 228 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRGGSGGGGSGGGGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 120 - protein, 229 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRGGSGGGGSGGGGSDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 121 - protein, 225 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRGGSGGGGSGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 122 - protein, 226 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRGGSGGGGSGGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 123 - protein, 227 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRGGSGGGGSGGGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 124 - protein, 223 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRTVAAPSVDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 125 - protein, 224 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRGGSGGGGSDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 126 - protein, 222 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRGGSGGGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 127 - protein, 224 aa, Artificial Sequence
  Description of Artificial Sequence Synthetic polypeptide
DIQMTQSPSSLSASVGDRVTITCQASQDIDMDLNWYQQKPGKAPKLLISQANTLPPGVPSRFSGSGSGTDFTFTISSLQPEDFATYYCLQSDWLPLTFGQGTKLEIKRTVAAPSVFDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 128 - protein, 221 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRGGSGGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 129 - protein, 223 aa, Artificial Sequence
  Description of Artificial Sequence Synthetic polypeptide
DIQMTQSPSSLSASVGDRVTITCQASQDIDMDLNWYQQKPGKAPKLLISQANTLPPGVPSRFSGSGSGTDFTFTISSLQPEDFATYYCLQSDWLPLTFGQGTKLEIKRGGSGGGGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 130 - protein, 226 aa, Artificial Sequence
  Description of Artificial Sequence Synthetic polypeptide
DIQMTQSPSSLSASVGDRVTITCQASQDIDMDMNWYQQKPGKAPKLLISQANTLPPGVHSRFSGSGSGTDFTFTISSLQPEDFATYYCLQSDWLPLTFGQGTKLEIKRGGSGGGGSGGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 131 - protein, 228 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRTVAAPSVFIFPPDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 132 - protein, 221 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRTVAAPDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 133 - protein, 222 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRTVAAPSDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

(19) United States
(12) Patent Application Publication
(10) Pub. No.: US 2014/0235476 A1
(43) Pub. Date: Aug. 21, 2014

(54) MULTIVALENT BINDING PROTEIN COMPOSITIONS AND METHODS FOR IDENTIFYING VARIANTS OF SAME

(71) Applicant: AbbVie, Inc., Worcester, MA (US)

(72) Inventors: Jijie GU, Shrewsbury, MA (US); Maria Cristina HARRIS, Worcester, MA (US); Lorenzo BENATUIL, Northborough, MA (US); Chung-Ming HSIEH, Newton, MA (US)

(73) Assignee: AbbVie, Inc., Worcester, MA (US)

(21) Appl. No.: 14/141,501

(22) Filed: Dec. 27, 2013

Related U.S. Application Data
(60) Provisional application No. 61/746,663, filed on Dec. 28, 2012.
(60) Provisional application No. 61/746,629, filed on Dec. 28, 2012.

(51) Int. Cl. G01N 33/566; C07K 16/18

(52) U.S. Cl. CPC G01N 33/566; C07K 16/18
USPC 506/9; 506/18; 506/17; 506/14; 435/69.6; 530/387.3; 536/23.53; 435/254.21; 435/254.22; 435/254.23; 435/254.2

(57) ABSTRACT

Provided are protein, nucleic acid, and cellular libraries of multivalent binding proteins (e.g., DVD-Fab or DVD-Ig molecules) and the use of these libraries for the screening of multivalent binding proteins using cell surface display technology (e.g., yeast display).

### RELATED APPLICATIONS

This application is related to U.S. provisional application 61/746,629 filed on Dec. 28, 2012, and U.S. provisional application 61/746,663 filed on Dec. 28, 2012, which are both incorporated by reference herein in their entireties.

### BACKGROUND OF THE INVENTION

I. Field

The present disclosure pertains to methods and compositions for selecting multivalent binding proteins that specifically bind to one or more desired target antigens. More specifically, the disclosure relates to protein, nucleic acid, and cellular libraries of multivalent binding proteins (e.g., DVD-Fab or DVD-Ig molecules) and the use of these libraries for the screening of multivalent binding proteins using cell surface display technology (e.g., yeast display).

II. Description of Related Art

A wide variety of multispecific antibody formats have been developed (see Kriangkum, J., et al., Biomol Eng, 2001. 18(2): p. 31-40). Amongst them tandem single-chain Fv molecules and diabodies, and various derivatives there of, are the most widely used formats for the construction of recombinant bispecific antibodies. More recently diabodies have been fused to Fc to generate more Ig-like molecules, named di-diabodies (see Lu, D., et al., J Biol Chem, 2004. 279(4): p. 2856-65). In addition, multivalent antibody construct comprising two Fab repeats in the heavy chain of an IgG and capable of binding four antigen molecules has been described (see WO 0177342A1, and Miller, K., et al., J Immunol, 2003. 170(9): p. 4854-61).

Despite the many bispecific antibody formats available to the skilled artisan, there is often a need for the skilled artisan to improve the affinity of the bispecific antibody through affinity maturation. However, conventional affinity maturation approaches rely upon screening for affinity matured variants of the component binding domains of the multispecific antibody followed by their reassembly into the original multispecific format. Such reassembly often results in a loss of the desired improvement in binding affinity or other desirable binding characteristics. Accordingly, there is a need in the art for improved constructs, formats, and screening methodologies for identifying affinity variants of multivalent binding proteins in their desired multivalent format.

### SUMMARY OF THE INVENTION

The present invention provides a novel compositions and methods useful for the generation of improved multivalent binding proteins capable of binding two or more antigens simultaneously with high affinity.

Accordingly, in one aspect the invention provides a diverse library of binding proteins comprising a first polypeptide chain having the general formula VH1-(X1)n-VH2-C—(X2)n, wherein VH1 is a first heavy chain variable domain, X1 is a linker with the proviso that it is not a constant domain, VH2 is a second heavy chain variable domain, C is a heavy chain constant domain, X2 is a cell surface protein, and n is 0 or 1, and wherein the amino acid sequences of VH1, VH2 and/or X1 independently vary within the library.

In certain embodiments, the binding proteins further comprise a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional antigen binding sites.

In certain embodiments, the first and second polypeptide chains combine to form a DVD-Fab or a full length DVD-Ig. In certain embodiments, the first and second polypeptide chains combine to form a full length DVD-Ig.

In certain embodiments, the amino acid sequences of VL1, VL2 and/or Y1 independently vary within the library.

In certain embodiments, the amino acid sequences of at least one CDR of VH1, VH2, VL1 or VL2 independently varies within the library. In one embodiment, the amino acid sequences of HCDR3 of VH1, VH2 independently vary within the library. In one embodiment, the amino acid sequences of HCDR1 and HCDR2 of VH1 or VH2 independently vary within the library. In one embodiment, the amino acid sequences of HCDR1, HCDR2 and HCDR3 of VH1 or VH2 independently vary within the library. In one embodiment, the amino acid sequences of HCDR3 of VL1 or VL2 independently vary within the library. In one embodiment, the amino acid sequences of HCDR1 and HCDR2 of VL1 or VL2 independently vary within the library. In one embodiment, the amino acid sequences of HCDR1, HCDR2 and HCDR3 of VL1 or VL2 independently vary within the library.

In certain embodiments, X1 independently varies within the library and wherein X1 is selected from the amino acid sequences set forth in Table 7 and/or 11. In certain embodiments, Y1 independently varies within the library and wherein Y1 is selected from the amino acid sequences set forth in Table 7 and/or 11. In certain embodiments, X2 comprises the Aga2p polypeptide.

In certain embodiments, the library of binding proteins share at least 70, 75, 80, 85, 90, 95, 96, 97, 98, or 99 amino acid sequence identity with a reference binding protein. In certain embodiments, VH1 and VH2 of the reference binding protein specifically bind to different antigens.

In another aspect, the invention provides a diverse library of polynucleotides encoding the first and/or second polypeptide chains of a diverse library of binding proteins disclosed herein.

In another aspect, the invention provides a diverse library of expression vectors comprising the diverse library of polynucleotides disclosed herein.

In another aspect, the invention provides a library of transformed host cells, expressing a diverse library of binding proteins disclosed herein.

In certain embodiments, the binding proteins are anchored on the cell surface of the host cells. In one embodiment, the binding proteins are anchored on the cell surface through Aga1p.

In certain embodiments, the host cells are eukaryotic. In one embodiment, the host cells are yeast, e.g., Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus, Pichia pastoris, Rhodotorula rubra, Schizosaccharomyces pombe and Yarrowia lipolytica. In one embodiment, the host cells are Saccharomyces cerevisiae.

In another aspect, the invention provides a method of selecting a binding protein that specifically binds to a target antigen, the method comprising: providing a diverse library of transformed host cells expressing a diverse library of binding proteins disclosed herein; contacting the host cells with the target antigen; and selecting a host cell that bind to the target antigen, thereby identifying a binding protein that specifically binds to a target antigen.

In another aspect, the invention provides a method of selecting a binding protein that specifically binds to a first and a second target antigen simultaneously, the method comprising: providing a diverse library of transformed host cells expressing a diverse library of binding proteins disclosed herein; contacting the host cells with the first and second target antigen; and selecting a host cell that bind to the first and second target antigen, thereby identifying a binding protein that specifically binds to a first and a second target antigen simultaneously.

In certain embodiments of the methods of the invention, the host cells that bind to the first and/or second antigen are selected by Magnetic Activated Cell Sorting using magnetically labeled antigen. In certain embodiments of the methods of the invention, the host cells that bind to the first and/or second antigen are selected by Fluorescence Activated Cell Sorting using fluorescently labeled antigen.

In certain embodiments, the methods of the invention further comprise isolating the binding protein-encoding polynucleotide sequences from the selected host cells.

In another aspect, the invention provides a method of producing a binding protein comprising expressing in a host cell a binding protein that was selected using the methods disclosed herein.

In another aspect, the invention provides a multivalent binding protein having the general formula VH1-(X1)n-VH2-C—X2, wherein VH1 is a first heavy chain variable domain, X1 is a linker with the proviso that it is not a constant domain, VH2 is a second heavy chain variable domain, C is a heavy chain constant domain, X2 is an anchoring moiety, and n is 0 or 1.

In certain embodiments, the multivalent binding protein further comprises a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional antigen binding sites.

In certain embodiments, the binding protein is a DVD-Fab molecule. In certain embodiments, the binding protein is a full length DVD-Ig.

In certain embodiments, the anchoring moiety cell surface protein. In one embodiment, the anchoring moiety comprises the Aga2p polypeptide.

In another aspect, the invention provides a polynucleotide encoding a binding protein disclosed herein.

In another aspect, the invention provides a host cell expressing a binding protein disclosed herein.

### BRIEF DESCRIPTION OF THE DRAWING

FIG. 1 depicts exemplary multivalent binding protein formats and cellular display methods.

FIG. 2 is a schematic representation of an exemplary method of selecting for multivalent binding proteins using yeast cell surface display. Antigen-binding, binding protein-expressing yeast cells are selected by two rounds of MACS (Magnetic Activated Cell Sorting) and two rounds of FACS (Fluorescence Activated Cell Sorting).

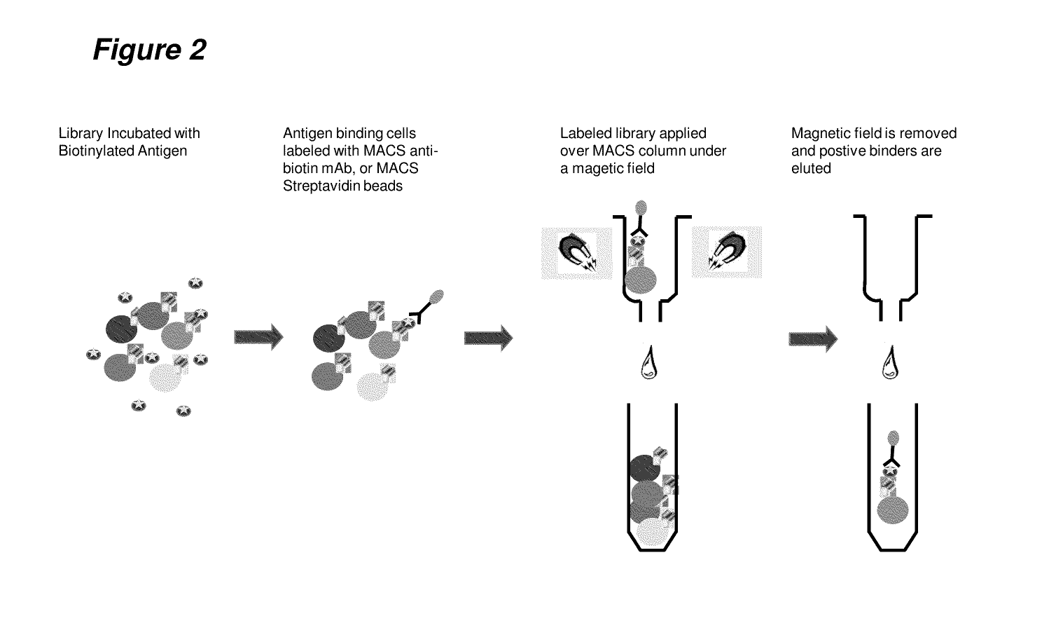

FIG. 3 is a schematic representation of an exemplary method for construction of a DVD-Fab yeast display library.

### DETAILED DESCRIPTION

The present invention provides a novel compositions and methods useful for the generation of improved multivalent binding proteins capable of binding two or more antigens simultaneously with high affinity.

### I. DEFINITIONS

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well known and commonly used in the art.

In order that the present invention may be more readily understood, certain terms are first defined.

The term “multivalent binding protein” is used throughout this specification to denote a binding protein comprising two or more antigen binding sites, each of which can bind independently bind to an antigen.

The terms “dual variable domain immunoglobulin” or “DVD-Ig” refer to the multivalent binding proteins disclosed in, e.g., U.S. Pat. No. 8,258,268, which is herein incorporated by reference in its entirety.

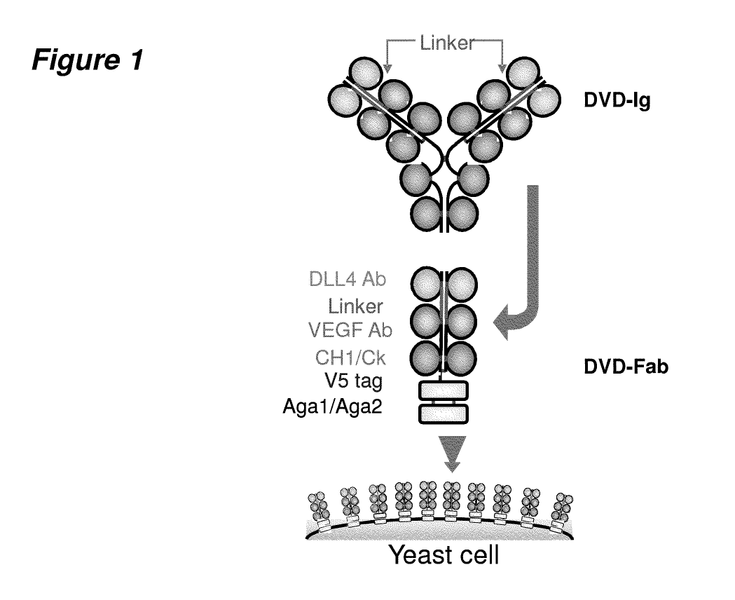

The term “DVD-Fab” refers to the antigen binding fragment of a DVD molecule that is analogous to an antibody Fab fragment. An exemplary DVD-Fab is depicted in FIG. 1 herein.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (Winter, et al. U.S. Pat. Nos. 5,648,260; 5,624,821). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to Fc.gamma.R5 and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region (Huber et al. Nature; 264: 415-20; Thies et al 1999 J Mol Biol; 293: 67-79.). Mutation of cysteine residues within the hinge regions to prevent heavy chain-heavy chain disulfide bonds will destabilize dimeration of CH3 domains. Residues responsible for CH3 dimerization have been identified (Dall'Acqua 1998 Biochemistry 37: 9266-73.). Therefore, it is possible to generate a monovalent half-Ig. Interestingly, these monovalent half Ig molecules have been found in nature for both IgG and IgA subclasses (Seligman 1978 Ann Immunol 129: 855-70; Biewenga et al 1983 Clin Exp Immunol 51: 395-400). The stoichiometry of FcRn: Ig Fc region has been determined to be 2:1 (West et al 2000 Biochemistry 39: 9698-708), and half Fc is sufficient for mediating FcRn binding (Kim et al 1994 Eur J Immunol; 24: 542-548.). Mutations to disrupt the dimerization of CH3 domain may not have greater adverse effect on its FcRn binding as the residues important for CH3 dimerization are located on the inner interface of CH3 b sheet structure, whereas the region responsible for FcRn binding is located on the outside interface of CH2-CH3 domains. However the half Ig molecule may have certain advantage in tissue penetration due to its smaller size than that of a regular antibody. In one embodiment at least one amino acid residue is replaced in the constant region of the binding protein of the invention, for example the Fc region, such that the dimerization of the heavy chains is disrupted, resulting in half DVD Ig molecules.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′).sub.2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5). In addition single chain antibodies also include “linear antibodies” comprising a pair of tandem Fv segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

As used herein, the terms “VH domain” and “VL domain” refer to single antibody variable heavy and light domains, respectively, comprising FR (Framework Regions) 1, 2, 3 and 4 and CDR (Complementary Determinant Regions) 1, 2 and 3 (see Kabat et al. (1991) Sequences of Proteins of Immunological Interest. (NIH Publication No. 91-3242, Bethesda).

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

As used herein the term “framework (FR) amino acid residues” refers to those amino acids in the framework region of an immunogobulin chain. The term “framework region” or “FR region” as used herein, includes the amino acid residues that are part of the variable region, but are not part of the CDRs (e.g., using the Kabat definition of CDRs).

As used herein, the term “specifically binds to” refers to the ability of a binding polypeptide to bind to an antigen with an Kd of at least about 1×10^{˜6} M, 1×10⁻⁷ M, 1×10⁻⁸ M, 1×10⁻⁹ M, 1×10⁻¹⁰ M, 1×10⁻¹¹ M, 1×10⁻¹² M, or more, and/or bind to an antigen with an affinity that is at least two-fold greater than its affinity for a nonspecific antigen. It shall be understood, however, that the binding polypeptide are capable of specifically binding to two or more antigens which are related in sequence. For example, the binding polypeptides of the invention can specifically bind to both human and a non-human (e.g., mouse or non-human primate) orthologos of an antigen.

The term “Polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “linker” is used to denote polypeptides comprising two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). Preferred linkers include, but are not limited to, the amino acid linkers set forth in Table 7 and/or 11 herein.

The term “Kₒₙ”, as used herein, is intended to refer to the on rate constant for association of an antibody to the antigen to form the antibody/antigen complex as is known in the art.

The term “K_{off}”, as used herein, is intended to refer to the off rate constant for dissociation of an antibody from the antibody/antigen complex as is known in the art.

The term “Kd”, as used herein, is intended to refer to the dissociation constant of a particular antibody-antigen interaction as is known in the art.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. Most preferably host cells include but are not limited to the prokaryotic cell line E. Coli; mammalian cell lines CHO, HEK 293 and COS; the insect cell line Sf9; and the fungal cell Saccharomyces cerevisiae.

### II. MULTIVALENT BINDING PROTEINS

In one aspect, the invention provides multivalent binding proteins that can bind to two antigen simultaneously. These binding proteins generally comprise a first polypeptide chain having the general formula VH1-(X1)n-VH2-C—(X2)n, wherein VH1 is a first heavy chain variable domain, X1 is a linker with the proviso that it is not a constant domain, VH2 is a second heavy chain variable domain, C is a heavy chain constant domain, X2 is a cell surface protein, and n is 0 or 1. The binding proteins can also comprise a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional binding sites.

In certain embodiments, the multivalent binding proteins are dual variable domain immunoglobulin (DVD-Ig) molecules, or fragments thereof (e.g., DVD-Fab fragments) (see FIG. 1). Such DVD-Ig molecules comprise at least one heavy chain and at least one light a chain. The heavy chain comprises two different heavy chain variable domains (VH) linked in tandem (directly or via a short linker) by recombinant DNA techniques, followed by the constant domain CH1 and Fc region. Similarly, the light chain comprises two different light chain variable domains (VL) from the two different parent monoclonal antibodies linked in tandem (directly or via a short linker) by recombinant DNA techniques, followed by the light chain constant domain.

The variable domains can be obtained using recombinant DNA techniques from a parent antibody generated by any method known in the art. In a certain embodiments, the variable domain is a murine heavy or light chain variable domain. In a certain embodiments, the variable domain is a CDR grafted or a humanized variable heavy or light chain domain. In a certain embodiments, the variable domain is a human heavy or light chain variable domain.

In certain embodiments, the first and second variable domains are linked directly to each other using recombinant DNA techniques. In certain embodiments, the variable domains are linked via a linker sequence. Preferably two variable domains are linked. Three or more variable domains may also be linked directly or via a linker sequence. The variable domains may bind the same antigen or may bind different antigens. DVD molecules of the invention may include one immunoglobulin variable domain and one non-immunoglobulin variable domain such as ligand binding domain of a receptor, active domain of an enzyme. DVD molecules may also comprise two or more non-Ig domains.

The linker sequence may be a single amino acid or a polypeptide sequence. Preferably the linker sequences are selected from the group consisting of consisting of the amino acid sequences set forth in Table 7 and/or 11.

In certain embodiments, a constant domain is linked to the two linked variable domains using recombinant DNA techniques. In certain embodiments, heavy chain variable domains are linked to a heavy chain constant domain and light chain variable domains are linked to a light chain constant domain. In certain embodiments, the constant domains are human heavy chain constant domain and human light chain constant domain respectively. In certain embodiments, the DVD heavy chain is further linked to an Fc region. The Fc region may be a native sequence Fc region, or a variant Fc region. In certain embodiments, the Fc region is a human Fc region. In a preferred embodiment the Fc region includes Fc region from IgG1, IgG2, IgG3, IgG4, IgA, IgM, IgE, or IgD.

In certain embodiments, two heavy chain DVD polypeptides and two light chain DVD polypeptides are combined to form a DVD-Ig molecule. In certain embodiments, one DVD light chain and one DVD heavy chain (devoid of Fc region) are combined to form a DVD-Fab.

### III. LIBRARIES OF MULTIVALENT BINDING PROTEIN

In one aspect, the invention provides libraries of multivalent binding proteins (e.g., DVD-Ig molecules, (e.g., DVD-Fab molecules)). Such libraries are particularly useful for selecting multivalent binding proteins with improved properties relative to a reference binding molecule (e.g., improved binding kinetics or thermostability).

In certain embodiments, the library of binding proteins comprises a first polypeptide chain having the general formula VH1-(X1)n-VH2-C—(X2)n, wherein VH1 is a first heavy chain variable domain, X1 is a linker with the proviso that it is not a constant domain, VH2 is a second heavy chain variable domain, C is a heavy chain constant domain, X2 is a cell surface protein, and n is 0 or 1, and wherein the amino acid sequences of VH1, VH2 and/or X1 independently vary within the library. In one embodiment, the first polypeptide chain is a DVD-Ig heavy chain or a fragment thereof (e.g., a DVD-Fab heavy chain).

In certain embodiments, the binding proteins further comprise a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional binding sites. In one embodiment, the amino acid sequences of VL1, VL2 and/or Y1 independently vary within the library. In one embodiment, the second polypeptide chain is a DVD-Ig light chain or a fragment thereof (e.g., a DVD-Fab light chain).

Any region of the first or second polypeptide chains can be varied independently in the libraries of the invention. In certain embodiments, the amino acid sequences of at least one CDR of VH1, VH2, VL1 or VL2 independently varies within the library. In one embodiment, the amino acid sequences of HCDR3 of VH1, VH2 independently vary within the library. In one embodiment, the amino acid sequences of HCDR1 and HCDR2 of VH1 or VH2 independently vary within the library. In one embodiment, the amino acid sequences of HCDR1, HCDR2 and HCDR3 of VH1 or VH2 independently vary within the library. In one embodiment, the amino acid sequences of HCDR3 of VL1 or VL2 independently vary within the library. In one embodiment, the amino acid sequences of HCDR1 and HCDR2 of VL1 or VL2 independently vary within the library. In one embodiment, the amino acid sequences of HCDR1, HCDR2 and HCDR3 of VL1 or VL2 independently vary within the library.

The linker regions X1 and/or Y1 can be also be varied independently in the libraries of the invention. Any length and sequence of linkers can be employed. Suitable amino acid sequences for use in linker X1 and/or Y1 are set forth in Table 7 and/or 11 herein.

In certain embodiments, the libraries of the invention are used in cell surface display techniques (e.g., yeast display as described in Wittrup, et al. U.S. Pat. No. 6,699,658, incorporated herein by reference). Accordingly, in certain embodiments X2 comprises a cell surface anchor. Any molecule that can display the binding proteins on the surface of a cell can be employed in the invention including, without limitation, cell surface protein and lipids. In one embodiment, X2 comprises the Aga2p polypeptide and allows display of the binding protein on the surface of yeast that express the Aga1p polypeptide.

In certain embodiments, the library of binding proteins are employed to affinity mature a reference binding protein (e.g., DVD-Fab). Accordingly, in certain embodiments, the library of binding proteins share at least 70, 75, 80, 85, 90, 95, 96, 97, 98, or 99 amino acid sequence identity with a reference binding protein (e.g., DVD-Fab). In certain embodiments, the VH1 and VH2 of the reference binding protein specifically bind to different antigens.

In another aspect, the invention provides libraries of polynucleotides encoding the first and/or second polypeptide chains of the diverse library of binding proteins. The libraries can be produced by any art recognized means. In certain embodiments, the libraries are produced by combining portions of other libraries by overlap PCR In certain embodiments, libraries are produced by combining portions of other libraries by gap repair transformation in yeast cells. In certain embodiments, the nucleic acids encoding the binding proteins are operably linked to one or more expression control elements (e.g., promoters or enhancer elements).

In another aspect, the invention provides libraries of expression vectors comprising the diverse library of polynucleotides disclosed herein. In certain embodiments, the vectors comprise only a single chain (e.g., a light or a heavy chain) of the binding proteins disclosed herein. In certain embodiments, the vectors comprise both chains of the binding proteins. The two chains can be expressed separately from different promoters. Alternatively, the two chains can be expressed together as a bi-cistronic message from a single promoter.

In another aspect, the invention provides a library of transformed host cells, expressing the diverse library of binding proteins disclosed herein. In certain embodiments, the individual transformed cells in the library of transformed host cells express only one species from the diverse library binding proteins.

Any cells, prokaryotic or eukaryotic, are suitable for use as host cells. In certain embodiments, the host cells are yeast including, without limitation, Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus, Pichia pastoris, Rhodotorula rubra, Schizosaccharomyces pombe and Yarrowia lipolytica.

In certain embodiments, the expressed binding proteins are anchored on the surface of the host cell. Any means for anchoring can be employed in the invention. In certain embodiments, the binding proteins are anchored on the cell surface through Aga1p. This is usually achieved by the fusion of the Aga2p protein to one or more chain of the binding protein.

### IV. MULTIVALENT BINDING PROTEIN SCREENING METHODS

In another aspect, the invention provides a method of selecting a binding protein (e.g., a DVD-Fab) that specifically binds to a target antigen. The method generally comprises: a) providing a diverse library of transformed host cells expressing a diverse library of binding proteins disclosed herein; b) contacting the host cells with the target antigen; and c) selecting a host cell that bind to the target antigen, thereby identifying a binding protein that specifically binds to a target antigen.

In another aspect, the invention provides a method of selecting a binding protein that specifically binds to a first and a second target antigen simultaneously. The method generally comprises: a) providing a diverse library of transformed host cells expressing a diverse library of binding proteins disclosed herein; b) contacting the host cells with the first and second target antigen; and c) selecting a host cell that bind to the first and second target antigen, thereby identifying a binding protein that specifically binds to a first and a second target antigen simultaneously.

In certain embodiments of the foregoing methods, host cells that bind to the first and/or second antigen are selected by Magnetic Activated Cell Sorting using magnetically labeled antigen. In certain embodiments of the foregoing methods, host cells that bind to the first and/or second antigen are selected by Fluorescence Activated Cell Sorting using fluorescently labeled antigen.

Any host cells, prokaryotic or eukaryotic, are suitable for use in the foregoing methods. In certain embodiments, the host cells are yeast including, without limitation, Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus, Pichia pastoris, Rhodotorula rubra, Schizosaccharomyces pombe and Yarrowia lipolytica.

In certain embodiments, the expressed binding proteins are anchored on the surface of the host cell. Any means for anchoring can be employed in the invention. In certain embodiments, the binding proteins are anchored on the cell surface through Aga1p. This is usually achieved by the fusion of the Aga2p protein to one or more chain of the binding protein.

After selection of antigen-binding host cells, the polynucleotides encoding the binding proteins expressed by those cells can be isolated using any standard molecular biological means. These polynucleotides can be isolated and re-expressed in another cellular or acellular system as desired. Alternatively, these polynucleotides can be further modified and screened using the methods disclosed herein. In certain embodiments, the isolated polynucleotides are recombined with other polynucleotides (including libraries disclosed herein) to produce new, hybrid polynucleotides encoding novel binding proteins.

In certain embodiments, multiple diverse libraries are created, where each library contains clones that vary at a different discreet region of a reference binding protein. Each library is then screened separately for binding to the desired antigen(s) and the selected clones from each library are recombined to from a new library for screening. For example, to facilitate the affinity maturation of a reference binding protein, two distinct, diverse libraries can be created: a first diverse library in which only the HCDR1 and HCDR2 regions of a reference antibody are varied; and a second diverse library in which only the HCDR3 region of a reference antibody are varied. The first and the second library can be screened using the methods disclosed herein (e.g., using yeast display) to identify binding molecules with improved antigen binding characteristics. The polynucleotides encoding the selected binding proteins can then be recombined (e.g., by overlap PCR or yeast GAP repair) to form a third library comprising the HCDR1 and HCDR2 regions from the first library and the HCDR3 regions form second library. This third library can then be screened using the methods disclosed herein to identify binding proteins with further improved antigen binding characteristics.

Binding proteins selected using the methods disclosed herein can be isolated and re-expressed in another cellular or acellular system as desired.

### V. ENGINEERED MULTIVALENT BINDING PROTEINS

In certain preferred embodiments, the multivalent binding proteins produced using the methods and compositons disclosed herein exhibit improved properties (e.g., affinity or stability) with respect to a corresponding parental reference binding protein. For example, the engineered binding protein may dissociate from its target antigen with a k_{off} rate constant of about 0.1 s⁻¹ or less, as determined by surface plasmon resonance, or inhibit the activity of the target antigen with an IC₅₀ of about 1×10⁻⁶M or less. Alternatively, the binding protein may dissociate from the target antigen with a k_{off} rate constant of about 1×10⁻² s⁻¹ or less, as determined by surface plasmon resonance, or may inhibit activity of the target antigen with an IC₅₀ of about 1×10⁻⁷ M or less. Alternatively, the binding protein may dissociate from the target with a k_{off} rate constant of about 1×10⁻³ s⁻¹ or less, as determined by surface plasmon resonance, or may inhibit the target with an IC₅₀ of about 1×10⁻⁸ M or less. Alternatively, binding protein may dissociate from the target with a k_{off} rate constant of about 1×10⁻⁴ s⁻¹ or less, as determined by surface plasmon resonance, or may inhibit its activity with an IC₅₀ of about 1×10⁻⁹M or less. Alternatively, binding protein may dissociate from the target with a k_{off} rate constant of about 1×10⁻⁵s⁻¹ or less, as determined by surface plasmon resonance, or inhibit its activity with an IC₅₀ of about 1×10⁻¹⁰ M or less. Alternatively, binding protein may dissociate from the target with a k_{off} rate constant of about 1×10⁻⁵s⁻¹ or less, as determined by surface plasmon resonance, or may inhibit its activity with an IC₅₀ of about 1×10⁻¹¹ M or less.

In certain embodiments, the engineered binding protein comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the binding protein can comprise a light chain constant region, either a kappa light chain constant region or a lambda light chain constant region. The binding protein comprises a kappa light chain constant region. Alternatively, the binding protein portion can be, for example, a Fab fragment or a single chain Fv fragment.

In certain embodiments, the engineered binding protein comprises an engineered effector function known in the art (see, e.g., Winter, et al. U.S. Pat. Nos. 5,648,260; 5,624,821). The Fc portion of a binding protein mediates several important effector functions e.g. cytokine induction, ADCC, phagocytosis, complement dependent cytotoxicity (CDC) and half-life/clearance rate of binding protein and antigen-binding protein complexes. In some cases these effector functions are desirable for therapeutic binding protein but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of binding proteins. In still another embodiment at least one amino acid residue is replaced in the constant region of the binding protein, for example the Fc region of the binding protein, such that effector functions of the binding protein are altered.

In certain embodiments, the engineered binding protein is derivatized or linked to another functional molecule (e.g., another peptide or protein). For example, a labeled binding protein of the invention can be derived by functionally linking a binding protein or binding protein portion of the invention (by chemical coupling, genetic fusion, noncovalent association or otherwise) to one or more other molecular entities, such as another binding protein (e.g., a bispecific binding protein or a diabody), a detectable agent, a cytotoxic agent, a pharmaceutical agent, and/or a protein or peptide that can mediate associate of the binding protein with another molecule (such as a streptavidin core region or a polyhistidine tag).

Useful detectable agents with which a binding protein or binding protein portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. A binding protein may also be derivatized with detectable enzymes, such as alkaline phosphatase, horseradish peroxidase, glucose oxidase and the like. When a binding protein is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. A binding protein may also be derivatized with biotin, and detected through indirect measurement of avidin or streptavidin binding.

In other embodiment, the engineered binding protein is further modified to generate glycosylation site mutants in which the O- or N-linked glycosylation site of the binding protein has been mutated. One skilled in the art can generate such mutants using standard well-known technologies. Glycosylation site mutants that retain the biological activity, but have increased or decreased binding activity, are another object of the present invention.

In still another embodiment, the glycosylation of the engineered binding protein or antigen-binding portion of the invention is modified. For example, an aglycoslated binding protein can be made (i.e., the binding protein lacks glycosylation). Glycosylation can be altered to, for example, increase the affinity of the binding protein for antigen. Such carbohydrate modifications can be accomplished by, for example, altering one or more sites of glycosylation within the binding protein sequence. For example, one or more amino acid substitutions can be made that result in elimination of one or more variable region glycosylation sites to thereby eliminate glycosylation at that site. Such aglycosylation may increase the affinity of the binding protein for antigen. Such an approach is described in further detail in PCT Publication WO2003016466A2, and U.S. Pat. Nos. 5,714,350 and 6,350,861, each of which is incorporated herein by reference in its entirety.

Additionally or alternatively, an engineered binding protein of the invention can be further modified with an altered type of glycosylation, such as a hypofucosylated binding protein having reduced amounts of fucosyl residues or a binding protein having increased bisecting GlcNAc structures. Such altered glycosylation patterns have been demonstrated to increase the ADCC ability of binding proteins. Such carbohydrate modifications can be accomplished by, for example, expressing the binding protein in a host cell with altered glycosylation machinery. Cells with altered glycosylation machinery have been described in the art and can be used as host cells in which to express recombinant binding proteins of the invention to thereby produce a binding protein with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342 80, each of which is incorporated herein by reference in its entirety. Using techniques known in the art a practitioner may generate binding proteins exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. patent Publication Nos. 20040018590 and 20020137134 and PCT publication WO2005100584 A2).

### VI. PRODUCTION OF MULTIVALENT BINDING PROTEINS

Engineered binding proteins of the present invention may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the binding proteins of the invention in either prokaryotic or eukaryotic host cells, expression of binding proteins in eukaryotic cells is preferable, and most preferable in mammalian host cells, because such eukaryotic cells (and in particular mammalian cells) are more likely than prokaryotic cells to assemble and secrete a properly folded and immunologically active binding protein.

Preferred mammalian host cells for expressing the recombinant binding proteins of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding binding protein genes are introduced into mammalian host cells, the binding proteins are produced by culturing the host cells for a period of time sufficient to allow for expression of the binding protein in the host cells or, more preferably, secretion of the binding protein into the culture medium in which the host cells are grown. Binding proteins can be recovered from the culture medium using standard protein purification methods.

Host cells can also be used to produce functional binding protein fragments, such as Fab fragments or scFv molecules. It will be understood that variations on the above procedure are within the scope of the present invention. For example, it may be desirable to transfect a host cell with DNA encoding functional fragments of either the light chain and/or the heavy chain of a binding protein of this invention. Recombinant DNA technology may also be used to remove some, or all, of the DNA encoding either or both of the light and heavy chains that is not necessary for binding to the antigens of interest. The molecules expressed from such truncated DNA molecules are also encompassed by the binding proteins of the invention. In addition, bifunctional binding proteins may be produced in which one heavy and one light chain are a binding protein of the invention and the other heavy and light chain are specific for an antigen other than the antigens of interest by crosslinking a binding protein of the invention to a second binding protein by standard chemical crosslinking methods.

In a preferred system for recombinant expression of a binding protein, or antigen-binding portion thereof, of the invention, a recombinant expression vector encoding both the binding protein heavy chain and the binding protein light chain is introduced into dhfr-CHO cells by calcium phosphate-mediated transfection. Within the recombinant expression vector, the binding protein heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the binding protein heavy and light chains and intact binding protein is recovered from the culture medium. Standard molecular biology techniques are used to prepare the recombinant expression vector, transfect the host cells, select for transformants, culture the host cells and recover the binding protein from the culture medium. Still further the invention provides a method of synthesizing a recombinant binding protein of the invention by culturing a host cell of the invention in a suitable culture medium until a recombinant binding protein of the invention is synthesized. The method can further comprise isolating the recombinant binding protein from the culture medium.

### VII. EXEMPLIFICATION

The present invention is further illustrated by the following examples which should not be construed as further limiting. The contents of Sequence Listing, figures and all references, patents and published patent applications cited throughout this application are expressly incorporated herein by reference.

### Example 1

### Construction of DVD-Fab Yeast Display Vector

A DLL4/VEGF DVD-Fab (comprising the VH and VL domains of anti-DLL4 clone h1A11.1 and an anti-VEGF antibody) was cloned into the yeast expression vector pFabB in a multiple step process. Briefly, the VH coding region of h1A11.1-short-Anti-VEGF was amplified from a different expression vector by PCR and inserted into pFabB vector (linearized with SpeI and SalI) by homologous recombination. The Vk coding region of h1A11.1-short-anti-VEGF was similarly amplified using 2-step overlapping PCR. The first PCR step amplified the h1A11.1-short-Anti-VEGF Vk region from a different expression vector, the second PCR step amplified the GAS leader sequence. The overlapping PCR product was then inserted into pFabB vector linearized with BamHI and BsiWI, containing the h1A11.1-short-Anti-VEGF VH correct sequence, by homologous recombination. After sequence confirmation, the pFabB-h1A11.1-SS-Anti-VEGF vector was transformed into chemically competent S. cerevisiae cells.

Upon induction of the cells, stainings were performed to confirm binding of the surface-expressed h1A11.1-SS-VEGF DVD-Fab to both DLL4 (human and murine) and VEGF. Expression of heavy and light chain on the surface of yeast was determined to be about 60%. After incubation of the cells with antigen for 1 h at 37 C, binding to huDLL4 and muDLL4 at 100 nM was observed and of VEGF-Alexa647 at 300 nM.

### Example 2

### Design and Construction of h1A11.1/VEGF DVD-Fab Library for Outer Domain Affinity Maturation

Sequence alignment showed that the DLL4 antibody h1A11.1 shares the highest identity to human germlines VH3-7/JH4 and O2/JK2. Based on previous affinity maturation of mAb h1A11.1, only VH-CDR1 and VH-CDR2 were mutagenized. The h1A11.1 VH-CDR3 and VK sequences were left unchanged. To improve the affinity of h1A11.1 to DLL4, hypermutated CDR residues were identified from other human antibody sequences in the IgBLAST database that also shared high identity to germlines VH3-7. The corresponding h1A11.1 CDR residues were then subjected to limited mutagenesis by PCR with primers having low degeneracy at these positions to create one antibody library in the DVD-Ig Fab format suitable for use in affinity maturation procedure. The library contained mutations at residues 30, 31, 32, 35, 50, 52, 52a, 55, 56, 57 and 58 in the VH CDR1 and 2 (Kabat numbering). To further increase the identity of h1A11.1 to the human germline framework sequences, a binary degeneracy at VH position 76 (S/N) was introduced into the library (see Table 1). To construct the library for h1A11.1/VEGF VH multiple steps of overlapping PCR were performed using doped primers to introduce mutations in VH-CDR1 and VH-CDR2 of h1A11.1. The final library contained short linkers to separate the DLL4 and VEGF variable domains (short linker VH sequence=ASTKGP; short linker VL sequence=TVAAP). The derived h1A11.1/VEGF VH PCR product was introduced into pFabB previously linearized with SpeI and SalI and containing h1A11.1/VEGF Vk coding sequence.

### Example 3

### Sorting h1A11.1/VEGF DVD-Fab Yeast Display Library

The h1A11.1/VEGF DVD-Fab library described in Example 2 was transformed into EBY100 yeast cells and the library size determined to be 1.3×10⁹. It was then displayed on the yeast cell surface and selected against DLL4 extracellular domain and VEGF by magnetic activated cell sorting (MACS) then fluorescence activated cell sorting (FACS). Two rounds of MACS were carried out by oversampling the cells 10 folds and by using a 10-fold antigen excess. Similar conditions were used for the three rounds of sorting. Sorting was done by dual labeling of library cells, gating on the best DLL4 expressors and binders and by collecting the best simultaneous binders to DLL4 and VEGF. Conditions for MACS and FACS sorting are described in Table 2 where M=MACS and S=FACS sorting.

TABLE 1
Mutations in h1A11.1 VH Amino Acid Sequence for Outer
Domain Affinity Maturation of DLL4/VEGF DVD-Fab

```
Mutated h1A11.1 VH Sequence (SEQ ID NO:):
EVQLVESGGGLVQPGGSLRLSCAASGFTFSNFPMAWVRQAPGKGLEWVATISSSDGTTYY
                                                          RKL T S RW ANSF
                                                          NIY P K NS VRIC
                                                          KTV K I IL RMPS
                                                          IYI G P TY DKNN
                                                          GSR D A CR WIML
                                                          TRP V N FF SAAR
                                                          PPC S E WV CYKQ
                                                          MHS E R GT YSRP
                                                          LEN Q C DK QFCI
                                                          HDH N AG MV H
                                                          FCD M LP
                                                          D L L
                                                          C F G
                                                          A
RDSVKGRFTISRDNAKNSLYLQMNSLRAEDTAVYYCARGYYNSPFAYWGQGTLVTVSS
                                S
```

Selection for improved h1A11.1 affinity clones was carried out under the conditions set forth in Table 2 and amino acid sequences of affinity-modulated h1A11.1 clones were recovered for converting back to DVD-IgG format for further characterization (see Table 3). A total of 11 clones were identified through the second and third round of cell sorting, but only ten were converted to DVD-IgG format because clone h1A11.1-A02-53 had a cysteine in the CDR2.

TABLE 2
Conditions for MACS and FACS sorting of yeast expressing
h1A11.1/VEGF DVD-Fab libraries

| | | | | | Tem- |
|---|---|---|---|---|---|
| | | | | | per- |
| Sort | Library | Ag [Ag] | Time | | ature |
| M1 | h1A11.1 (H1 + H2) | 100 nM huDLL4-biotin | 1 | h | 37° C. |
| M2 | h1A11.1 (H1 + H2) | 100 nM huDLL4-biotin | 1 | h | 37° C. |
| M2S1 | h1A11.1 (H1 + H2) | 100 nM muDLL4- | 1 | h | 37° C. |
| | | Alexa647 | | | |
| | | 100 nM VEGF-biotin | | | |
| M2S2 | h1A11.1 (H1 + H2) | 25 nM muDLL4- | 15 | min. | 37° C. |
| | | Alexa647 | | | |
| | | 100 nM VEGF-biotin | | | |
| M2S3 | h1A11.1 (H1 + H2) | 3 nM muDLL4- | 5 | min. | RT |
| | | Alexa647 | | | |
| | | 300 nM VEGF-biotin | | | |

TABLE 3
Protein sequences of antibody clones identified from affinity
maturation library for anti-DLL4 antibody h1A11.1
Affinity Matured Clones: Heavy Chain (VH) Regions

| h1A11.1- | EVQLVESGGGLVQPGGSLRLSCAASGFTFSHFPMAWVRQAPGKGLEWVASI | | |
|---|---|---|---|
| B9-S2 | SSSDSTTNYRDSVKGRFTISRDNAKNSLYLQMNSLRAEDTAVYYCARGYYN | | |
| | SPFAYWGQGTLVTVSS (SEQ ID NO:) | | |
| | CDR1 | CDR2 | CDR3 |
| | SHFPMA | SISSSDSTTNYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFKNFPMAWVRQAPGKGLEWVATI | | |
| G10-S2 | SSSDLSTNYRDSVKGRFTISRDNAKNSLYLQMNSLRAEDTAVYYCARGYYN | | |
| | SPFAYWGQGTLVTVSS (SEQ ID NO:) | | |
| | CDR1 | CDR2 | CDR3 |
| | KNFPMA | TISSSDLSTNYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFRNFPMAWVRQAPGKGLEWVASI | | |
| H3-S2 | SSSDGTTNYRDSVKGRFTISRDNAKSSLYLQMNSLRAEDTAVYYCARGYYN | | |
| | SPFAYWGQGTLVTVSS (SEQ ID NO:) | | |
| | CDR1 | CDR2 | CDR3 |
| | RNFPMA | SISSSDGTTNYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFRHFPMTWVRQAPGKGLEWVASI | | |
| F7-S2 | SSSDGTINYRDSVKGRFTISRDNAKNSLYLQMNSLRAEDTAVYYCARGYYN | | |
| | SPFAYWGQGTLVTVSS (SEQ ID NO:) | | |
| | CDR1 | CDR2 | CDR3 |
| | RHFPMT | SISSSDGTINYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFRNFPMAWVRQAPGKGLEWVATI | | |
| C1-S2 | SSSDGTPAYRDSVKGRFTISRDNAKNSLYLQMNSLRAEDTAVYYCARGYYN | | |
| | SPFAYWGQGTLVTVSS (SEQ ID NO:) | | |
| | CDR1 | CDR2 | CDR3 |
| | RNFPMA | TISSSDGTPAYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFRYFPMAWVRQAPGKGLEWVAAI | | |
| F12-S2 | SGSDGTASYRDSVKGRFTISRDNAKNSLYLQMNSLRAEDTAVYYCARGYYN | | |
| | SPFAYWGQGTLVTVSS (SEQ ID NO:) | | |
| | CDR1 | CDR2 | CDR3 |
| | RYFPMA | AISGSDGTASYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFNHFPMAWVRQAPGKGLEWVATI | | |
| G07-S2 | SSSDWTPYYRDSVKGRFTISRDNAKNSLYLQMNSLRAEDTAVYYCARGYYN | | |
| | SPFAYWGQGTLVTVSS (SEQ ID NO:) | | |
| | CDR1 | CDR2 | CDR3 |
| | NHFPMA | TISSSDWTPYYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFQKYPMAWVRQAPGKGLEWVATI | | |
| A02-S3 | SCSDGITHYRDSVKGRFTISRDNAKNSLYLQMNSLRAEDTAVYYCARGYYN | | |
| | SPFAYWGQGTLVTVSS (SEQ ID NO:) | | |
| | CDR1 | CDR2 | CDR3 |
| | QKYPMA | TISCSDGITHYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFRHFPMAWVRQAPGKGLEWVATI | | |
| A04-S3 | SSSDGATYYRDSVKGRFTISRDNAKSSLYLQMNSLRAEDTAVYYCARGYYN | | |
| | SPFAYWGQGTLVTVSS (SEQ ID NO:) | | |
| | CDR1 | CDR2 | CDR3 |
| | RHFPMA | TISSSDGATYYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFRHFPMAWVRQAPGKGLEWVASI | | |
| A10-S3 | SSSDGTSNYRDSVKGRFTISRDNAKSSLYLQMNSLRAEDTAVYYCARGYYN | | |
| | SPFAYWGQGTLVTVSS (SEQ ID NO:) | | |
| | CDR1 | CDR2 | CDR3 |
| | RHFPMA | SISSSDGTSNYRDSVKG | GYYNSPFAY |
| | (SEQ ID NO:) | (SEQ ID NO:) | (SEQ ID NO:) |
| h1A11.1- | EVQLVESGGGLVQPGGSLRLSCAASGFTFGHFPMAWVRQAPGKGLEWVATI | | |
| E06-S3 | SSSDGATNYRDSVKGRFTISRDNAKNSLYLQMNSLRAEDTAVYYCARGYYN | | |
| | SPFAYWGQGTLVTVSS (SEQ ID NO:) | | |
| | CDR1 | CDR2 | CDR3 |
| | GHFPMA | TISSSDGATNYRDSVKG | GYYNSPFAY |
| | (SEQ ID NO:) | (SEQ ID NO:) | (SEQ ID NO:) |

S2 and S3 clones refer to clones identified after either two rounds or three rounds of sorting respectively.

### Example 4

### Characterization of DLL4/VEGF DVD-Fab Affinity Maturation Outputs

The affinity matured DLL4/VEGF clones identified and described in Table 3 were converted into full DVD-Ig molecules. Primers complementary to the 5′ and 3′ ends of each clone were designed and clones were amplified by PCR and introduced into the mammalian expression vector pHybE by homologous recombination. After performing bacterial colony PCR one clone of each construct was confirmed correct, scaled up and transiently transfected into HEK-293 cells for expression. Protein supernatants were harvested and purified by protein A affinity chromatography. Clone h1A11.1-E06-S3 was not purified because it expressed very poorly in HEK-293 cells. Purified material was utilized for characterization of DVD-Ig molecules by SEC, MS, stability assay (see Table 4) and Biacore (see Table 5 and Table 6). Stability assays were carried out at 50 mg/ml DVD-Ig in 15 mM histidine buffer (pH6.0) at 5° C. Monomer percentage was monitored at days 0, 8 and 21.

TABLE 4
SEC, MS and stability assay data for affinity matured
DLL4/VEGF clones

| | % | | Stability 5° C., |
|---|---|---|---|
| DVD clone | monomer | MS | 21 days* |
| h1A11.1-G10-S2- | 83.3 | OK | no loss of monomer |
| SS-Anti-VEGF | | | % |
| h1A11.1-F7-S2-SS- | 70.8 | OK | no loss of monomer |
| Anti-VEGF | | | % |
| h1A11.1-F12-S2-SS- | 63.6 | OK | no loss of monomer |
| Anti-VEGF | | | % |
| h1A11.1-C1-S2-SS- | 79.2 | OK | no loss of monomer |
| Anti-VEGF | | | % |
| h1A11.1-B9-S2-SS- | 70.5 | VL OK | no loss of monomer |
| Anti-VEGF | | VH 5.54 Da diff. | % |
| h1A11.1-H3-S2-SS- | 66.5 | OK | no loss of monomer |
| Anti-VEGF | | | % |
| h1A11.1-G7-S2-SS- | 73.2 | OK | no loss of monomer |
| Anti-VEGF | | | % |
| h1A11.1-A10-S3- | 63.3 | OK | no loss of monomer |
| SS-Anti-VEGF | | | % |
| h1A11.1-A04-S3- | 61.3 | OK | no loss of monomer |
| SS-Anti-VEGF | | | % |

*Samples h1A11.1-A10-S3-SS-Anti-VEGF and h1A11.1-A04-S3-SS-Anti-VEGF were tested at day 0, 7 and 47 days.

TABLE 5
Binding kinetics of DLL4/VEGF affinity maturation-derived
DVD-Ig molecules to huDLL4

| | | | | K_{D} ratio |
|---|---|---|---|---|
| | | | | (parental |
| | | | | DVD/AM |
| DVD clone | Ka | Kd | K_{D} | DVD) |
| h1A11.1-SS- | 1.33E+05 | 2.66E−03 | 2.00E−08 | 1.00 |
| Anti-VEGF | | | | |
| h1A11.1-G10- | 1.35E+05 | 7.45E−05 | 5.54E−010 | 36.024 |
| S2-SS-Anti- | | | | |
| VEGF | | | | |
| h1A11.1-F7-S2- | 5.7E+05 | 3.19E−04 | 5.56E−10 | 35.901 |
| SS-Anti-VEGF | | | | |
| h1A11.1-F12- | 1.60E+05 | 1.24E−04 | 7.76E−10 | 25.699 |
| S2-SS-Anti- | | | | |
| VEGF | | | | |
| h1A11.1-C1-S2- | 1.21E+05 | 1.10E−04 | 9.11E−10 | 21.911 |
| SS-Anti-VEGF | | | | |
| h1A11.1-B9-S2- | 1.15E+05 | 1.06E−04 | 9.23E−10 | 21.617 |
| SS-Anti-VEGF | | | | |
| h1A11.1-H3-S2- | 1.30E+05 | 1.36E−03 | 1.04E−09 | 19.128 |
| SS-Anti-VEGF | | | | |
| h1A11.1-G7-S2- | 1.17E+05 | 1.55E−04 | 1.32E−09 | 15.160 |
| SS-Anti-VEGF | | | | |
| h1A11.1-A10- | 1.34E+05 | 5.86E−05 | 4.39E−10 | 62.8 |
| S3-SL-Anti- | | | | |
| VEGF | | | | |
| h1A11.1-A04- | 1.25E+05 | 9.04E−05 | 7.23E−10 | 38.2 |
| S3-SL-Anti- | | | | |
| VEGF | | | | |

DVD = Dual Variable Domain Ig molecule;
E = multiply by 10 to indicated exponent;
Ka (M⁻¹s⁻¹);
Kd (s⁻¹);
K_{D} (M);
SS (short linker in both VH and VL variable regions);
samples h1A11.1-A10-S3-SL-Anti-VEGF and h1A11.1-A04-S3-SL-Anti-VEGF were tested with short long linkers (for VH and VL respectively) as opposed to short short linkers

TABLE 6
Binding kinetics of DLL4/VEGF affinity maturation-derived
DVD-Ig molecules to muDLL4

| | | | | K_{D} ratio |
|---|---|---|---|---|
| | | | | (parental |
| | | | | DVD/AM |
| DVD clone | Ka | Kd | K_{D} | DVD) |
| h1A11.1-SS- | 4.79E+05 | 1.03E−02 | 2.14E−08 | 1.00 |
| Anti-VEGF | | | | |
| h1A11.1-G10- | 2.07E+05 | 1.12E−04 | 5.39E−010 | 39.651 |
| S2-SS-Anti- | | | | |
| VEGF | | | | |
| h1A11.1-F7-S2- | 1.34E+06 | 4.83E−04 | 3.61E−10 | 59.192 |
| SS-Anti-VEGF | | | | |
| h1A11.1-F12- | 2.24E+05 | 1.70E−04 | 7.04E−10 | 30.378 |
| S2-SS-Anti- | | | | |
| VEGF | | | | |
| h1A11.1-C1-S2- | 1.86E+05 | 1.62E−04 | 8.70E−10 | 24.578 |
| SS-Anti-VEGF | | | | |
| h1A11.1-B9-S2- | 1.67E+05 | 2.05E−04 | 1.23E−09 | 17.396 |
| SS-Anti-VEGF | | | | |
| h1A11.1-H3-S2- | 1.97E+05 | 2.94E−04 | 1.49E−09 | 14.311 |
| SS-Anti-VEGF | | | | |
| h1A11.1-G7-S2- | 1.69E+05 | 2.66E−04 | 1.57E−09 | 13.618 |
| SS-Anti-VEGF | | | | |
| h1A11.1-A10- | 1.71E+05 | 1.33E−04 | 7.74E−10 | 53.8 |
| S3-SS-Anti- | | | | |
| VEGF | | | | |
| h1A11.1-A04- | 1.80E+05 | 8.12E−05 | 4.51E−10 | 92.2 |
| S3-SS-Anti- | | | | |
| VEGF | | | | |

### Example 5

### Design and Construction of DLL4/VEGF DVD-Fab Linker Library

A DLL4/VEGF linker library was constructed using 3 different types of linkers: standard long/short linkers, GS linkers and rigid linkers (see Table 7 and/or 11 for amino acid sequences of linkers). Oligonucleotides containing each DNA linker sequence with 5′ ends complementary to the DLL4 sequence of h1A11.1 and with 3′ ends complementary to the VEGF sequence of Anti-VEGF were synthesized. Oligonucleotides were pooled in equimolar amounts in 6 different groups based on their type and on their length. PCR reactions were carried out separately with the 6 different oligonucleotide groups using DLL4/VEGF M2S-encoding DNA isolated from previous DLL4/VEGF affinity maturation (see Example 3) as template. Reactions for VH and VL linker libraries were carried out separately. Each PCR product was gel purified, concentrated and mixed in equimolar amounts to result in one final PCR product containing the linker library for VH and for VL separately. The VH and VL-containing PCR products were then combined into one product by overlapping PCR and recombined into pFabB expression vector linearized with SpeI, SalI, BsiWI and BamHI by yeast electroporation. Different ratios of vector and insert were used (ug vector/ug insert=4/12, 4/18 and 4/24) and derived populations of yeast cells were grown separately first then eventually were combined together in a manner that allowed each population to be oversampled 10-fold. Yeast colony PCR was performed on the pooled populations to determine the diversity of the final library. After sequence analysis the size of the final DLL4 M2S 1 recombined linker library was determined to be 2.3×10⁷ and the linker distribution of each linker subtype followed the predicted distribution (see Table 8). It was also observed that about 66% of the clones had a combination of different types of linkers for VH and VL, while about 34% had a combination of the same type of linker

TABLE 7
Amino acid sequences of linkers used
for linker library construction

| | | SEQ | | SEQ |
|---|---|---|---|---|
| Linker | VH linker | ID | | ID |
| type | (name) | NO: | VL linker | NO: |
| Standard | ASTKGPSVFPLAP | | TVAAPSVFIFPP | |
| | (VH13) | | (VL12) | |
| Standard | ASTKGPSVFPLA | | TVAAPSVFIFP | |
| | (VH12) | | (VL11) | |
| Standard | ASTKGPSVFPL | | TVAAPSVFIF | |
| | (VH11) | | (VL10) | |
| Standard | ASTKGPSVFP | | TVAAPSVFI | |
| | (VH10) | | (VL9) | |
| Standard | ASTKGPSVF | | TVAAPSVF | |
| | (VH9) | | (VL8) | |
| Standard | ASTKGPSV | | TVAAPSV | |
| | (VH8) | | (VL7) | |
| Standard | ASTKGPS | | TVAAPS | |
| | (VH7) | | (VL6) | |
| Standard | ASTKGP | | TVAAP | |
| | (VH6) | | (VL5) | |
| GS | GGGGSGGGGSGGGG | | GGSGGGGSGGGGS | |
| | (GS14VH) | | (GS13VL) | |
| GS | GGGGSGGGGSGGG | | GGSGGGGSGGGG | |
| | (GS13VH) | | (GS12VL) | |
| GS | GGGGSGGGGSGG | | GGSGGGGSGGG | |
| | (GS12VH) | | (GS11VL) | |
| GS | GGGGSGGGGSG | | GGSGGGGSGG | |
| | (GS11VH) | | (GS10VL) | |
| GS | GGGGSGGGGS | | GGSGGGGSG | |
| | (GS10VH) | | (GS9VL) | |
| GS | GGGGSGGGG | | GGSGGGGS | |
| | (GS9VH) | | (GS8VL) | |
| GS | GGGGSGGG | | GGSGGGG | |
| | (GS8VH) | | (GS7VL) | |
| GS | GGGGSGG | | GGSGGG | |
| | (GS7VH) | | (GS6VL) | |
| GS | GGGGSG | | GGSGG | |
| | (GS6VH) | | (GS5VL) | |
| Rigid | TPAPLPAPLPAPTT | | TPAPLPAPLPAPT | |
| linker | (RL14VH) | | (RL13VL) | |
| Rigid | TPAPLPAPAPTT | | TPAPLPAPAPT | |
| linker | (RL12VH) | | (RL11VL) | |
| Rigid | TPAPLPAPTT | | TPAPLPAPT | |
| linker | (RL10VH) | | (RL9VL) | |
| Rigid | TPAPLPTT | | TPAPLPT | |
| linker | (RL8VH) | | (RL7VL) | |
| Rigid | TPAPTT | | TPAPT | |
| linker | (RL6VH) | | (RL5VL) | |

TABLE 8
Percentage linker distribution after linker library construction

| | VH linker | | VL linker | |
|---|---|---|---|---|
| Linker type | Predicted % | Actual % | Predicted % | Actual % |
| Regular | 36.4 | 37 | 36.4 | 36.5 |
| GS | 40.9 | 42 | 40.9 | 47 |
| Rigid | 22.7 | 21 | 22.7 | 16.5 |

### Example 6

### DLL4 M2S1/VEGF Recombined Linker Library Sorting

Scouting experiments were performed to determine optimal condition for library sorting. Suitable selective conditions were found to be 3 nM muDLL4 and 300 nM VEGF. The DLL4 M2S1/VEGF linker library was oversampled by 10-fold and labeling was done with 10-fold antigen excess as described in Example 3. Different labeling and sorting was performed under a variety of conditions (see Table 9). Antigen binding was carried out at 37° C. for 15 minutes. A total of 5 different outputs were collected.

TABLE 9
Labeling and sorting conditions of DLL4M2S1/VEGF
recombined linker library

| Library | Antigen [Ag] | Gate | Sort |
|---|---|---|---|
| DLL4 M2S1/VEGF | 3 nM | Best muDLL4 | 1-Best muDLL4 |
| rec. linker library | muDLL4 | binders | binders |
| DLL4 M2S1/VEGF | 3 nM | Best muDLL4 | 2-Best muDLL4 |
| rec. linker library | muDLL4 | binders | and VEGF |
| | 300 nM | | simultaneous |
| | VEGF | | binders |
| | | | 3-Best muDLL4 |
| | | | binders regardless |
| | | | of VEGF binding |
| DLL4 M2S1/VEGF | 3 nM | Best VEGF | 4-Best VEGF |
| rec. linker library | muDLL4 | binders | and muDLL4 |
| | 300 nM | | simultaneous |
| | VEGF | | binders |
| | | | 5-Best VEGF |
| | | | binders regardless |
| | | | of muDLL4 |
| | | | binding |

Upon sequence analysis of the 5 different outputs it was concluded that the best way to sort the library is to perform double staining and collect the best simultaneous binders (by gating on either DLL4 or VEGF best binders first). After another scouting experiment to determine the best antigen binding conditions for the 5 libraries, a second round of sorting was performed. Simultaneous binding of 0.3 nM muDLL4 and 100 nM VEGF was carried out at room temperature for 5 minutes. Only sorted populations 2, 4 and 5 from the first round (see Table 9) were sorted in the second round. Labeling and sorting conditions are set forth in Table 10.

TABLE 10
Labeling and sorting conditions of DLL4M2S2/VEGF
recombined linker library

| Library | Population | Antigen [Ag] | Gate | Sort |
|---|---|---|---|---|
| DLL4 | 2 | 0.3 nM | Best | Best muDLL4 |
| M2S2/VEGF | | muDLL4 | muDLL4 | and VEGF |
| rec. linker | | 100 nM | binders | simultaneous |
| library | | VEGF | | binders |
| DLL4 | 4 | 0.3 nM | Best VEGF | Best muDLL4 |
| M2S2/VEGF | | muDLL4 | binders | and VEGF |
| rec. linker | | 100 nM | | simultaneous |
| library | | VEGF | | binders |
| DLL4 | 5 | 100 nM | Best VEGF | Best VEGF |
| M2S2/VEGF | | VEGF | binders | binders |
| rec. linker | | | | |
| library | | | | |

A third round of sorting is performed, based upon the library diversity after the second round of sorting. Specifically, a scouting experiment is first performed as described herein (see Example 6) to determine optimal antigen concentrations and, based on that result, a third round of sorting is performed. Population 5 is gated as in the second round of sorting (see Table 10) to identify linker pairs that are best suited for inner domain (anti-VEGF in this case) affinity improvement, independent of DLL4 affinity. Populations 2 and 4 are gated as in the second round of sorting (see Table 10) to identify DLL4/VEGF DVD-Ig molecules with improved DLL4 binding and possibly VEGF binding. Output yeast cells are plated on SDCAA plates and 96 colonies are picked from each plate. Sequence analysis of all outputs is performed to determine the diversity of each population and which linker pairs are preferred for inner domain (VEGF) affinity improvement, outer domain (DLL4) affinity improvement by maintaining and/or improving affinity of inner domain (Anti-VEGF).

### Example 7

### Characterization of DLL4/VEGF DVD-Fab Recombinant Linker Library Output

The best performing DLL4/VEGF DVD-Fab recombinant linker library clones identified through several rounds of sorting are converted to DVD-Ig molecules and characterized as described in Example 4.

### Example 8

### Design and Construction of VEGF/DLL4 DVD-Fab Linker Library for Inner Domain Affinity Maturation

A VEGF/DLL4 linker library was constructed using 3 different types of linkers: standard long/short linkers, GS linkers and rigid linkers as in Example 5 (see Table 7 for amino acid sequences of linkers). Oligonucleotides containing each DNA linker sequence with 5′ ends complementary to the VEGF sequence of Anti-VEGF and with 3′ ends complementary to the DLL4 sequence of h1A11.1 were synthesized. Oligonucleotides were pooled in equimolar amounts in 6 different groups based on their type and on their length. PCR reactions were carried out separately with the 6 different oligonucleotide groups using pFabB-Anti-VEGF-GS14-h1A11.1 parental vector DNA as template. Reactions for VH and VL linker libraries were carried out separately. Each PCR product was gel purified and concentrated and mixed in equimolar amounts so that to have a one final PCR product containing the linker library for VH and for VL separately. The VH and VL-containing PCR products were then combined into one product by overlapping PCR and recombined into pFabB expression vector linearized with SpeI, SalI, BsiWI and BamHI by yeast electroporation. A ratio of ug vector/ug insert=4/12 was used and derived population of yeast cells was grown. Yeast colony PCR was performed on the population to determine the diversity of the final library. After sequence analysis the size of the final VEGF/DLL4 linker library was determined to be 3.5×10⁷ and all types of linkers were represented. After several rounds of sorting as described in Example 9, this library is recombined with h1A11.1 VH library for inner domain affinity maturation. This h1A11.1 VH library is designed as described in Example 2 and VEGF/DLL4 linker library-derived DNA are used as template for PCR. The derived VEGF/h1A11.1 VH PCR product are introduced into pFabB previously linearized with SpeI and SalI and containing VEGF/h1A11.1 Vk linker library coding sequence.

### Example 9

### Sorting VEGF/h1A11.1 DVD-Fab Yeast Display Linker Library and Recombined Library for Inner Domain (h1A11.1) Affinity Maturation

A VEGF/h1A11.1 DVD-Fab yeast display linker library is transformed into EBY100 yeast cells by electroporation and then displayed on cell surfaces and selected against DLL4 extracellular domain and VEGF by fluorescence activated cell sorting (FACS). Multiple rounds of sorting will be performed to reduce library diversity, in a similar manner to that set forth in Example 3. Specifically, sorting is performed by dual labeling of library cells, gating on the best DLL4 expressors and binders and by collecting the best simultaneous binders to DLL4 and VEGF. Selection for improved h1A11.1 affinity clones is then performed and amino acid sequences of affinity-modulated h1A11.1 clones are recovered for conversion to DVD-IgG format for further characterization.

### Example 10

### Characterization of VEGF/DLL4 DVD-Fab Affinity Maturation Output

Affinity matured VEGF/DLL4 clones are converted into full DVD-Ig molecules and characterized as described in Example 4.

### Example 11

### Apply Different Selection Conditions for DVD-Fab Yeast Library Sorting

A synthetic library of IL17/IL1α DVD-Fab is generated and recombined into pFabB yeast expression vectors by electroporation into yeast cells. Several IL17/IL1α DVD-Fab are selected based upon available data for multiple IL17/IL1α DVD-Ig molecules previously generated. These DVD-Ig molecules have been extensively characterized and have known binding affinities and potencies, solubility, stability and physicochemical properties. Several DVD-Ig molecules with good, acceptable and poor physicochemical properties are selected. These molecules are used as DNA template for PCR to construct the synthetic library. After being amplified they are mixed in equimolar amount before being transformed into yeast. The IL17/IL1α DVD-Fab library are selected using different conditions for sorting (salt concentration, buffer pH, different buffers, heating and possibly other methods). The selection pressure that allows selection of DVD-Ig molecules from the library with best physicochemical properties is determined. This method is optionally incorporated during affinity maturation of a DVD-Ig molecule to select not only for molecules with improved binding affinity but also with improved physicochemical properties.

### Example 12

### Design and Construction of IL1β/IL17 Mix and Match DVD-Fab Library

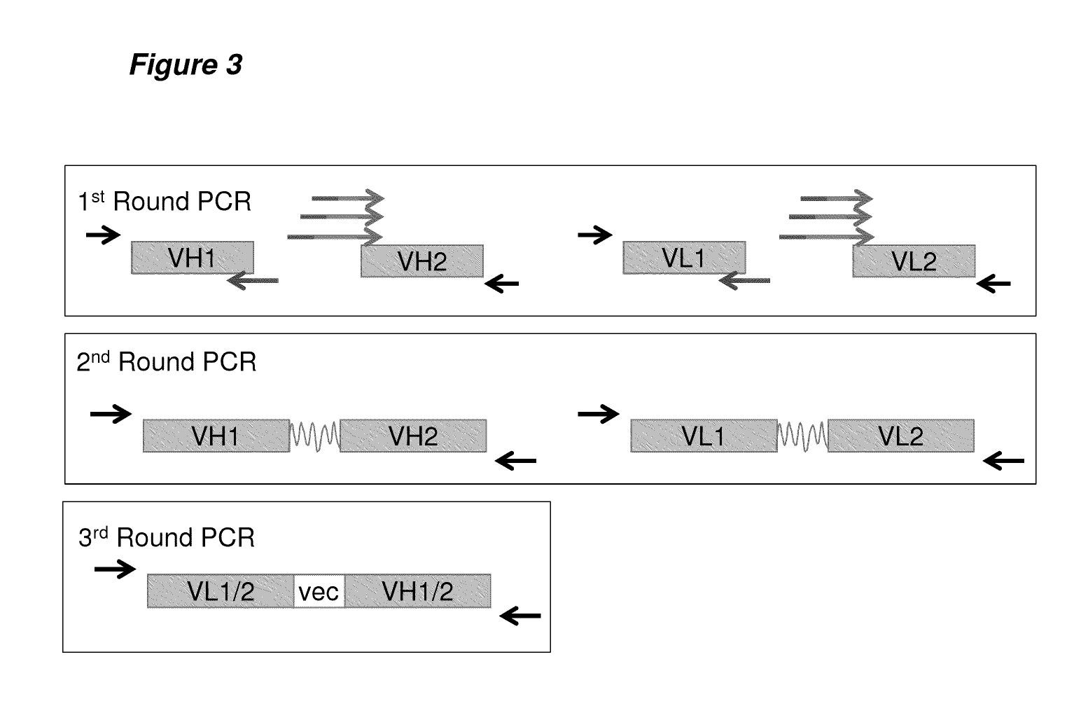

A IL1β/IL17 mix and match library was constructed using 7 outer domain mAbs to IL1β, 3 inner domains mAbs to IL17, and 2 types of linkers of various lengths (see Table 111). The library was constructed using an overlapping PCR strategy (see FIG. 3). Oligonucleotides were designed and synthesized in two groups: (1) reverse primers that anneals to the outer domain mAb sequence and encodes the DNA sequence of shortest linker length of a linker type (i.e. VH6); and (2) forward primers that anneal to the inner domain sequence and encode the DNA sequence of the entirety of the linker Each mAb VH and VL was PCR amplified separately using the appropriate primers; for the inner domains all primer oligonucleotides were pooled by type (i.e. all Elbow VH). Each PCR product was cleaned up using Qiagen QiaQuick PCR purification kit and then pooled in equal amounts grouped by mAb lineage and linker type for a total of 16 PCR pools for the second round PCR. For example four pools were created for the VH1 domain: 1B12 lineage with Elbow linker; 1B12 lineage with GS linker; E26 lineage with Elbow linker; and E26 lineage with GS linker Heavy chain and light chains were each assembled in four separate PCR reactions, for example: (1) 1B12 lineage+Elbow linkers+B6 lineage, (2) 1B12 lineage+Elbow linkers+10F7M11, (3) 1B12 lineage+GS linkers+B6 lineage, (4) 1B12 lineage+GS linkers+10F7M11. The second round PCR reactions were gel purified and equal amounts of heavy chain PCR, light chain PCR, and promoter sequence PCR were used for the third round PCR. The third round PCR product was gel purified, concentrated, and then recombined with linearized pFabB expression vector by yeast electroporation. The pFabB expression vector was linearized by digestion with SalI, BsiWI, and BamHI followed by gel purification and concentration. Based on dilution plating, the library size was estimated at 3×10⁸ members. After library yeast cells were grown, the library DNA was isolated from the yeast cells, transformed into E. coli, and colony PCR and sequencing performed to determine the distribution of the final library (see Table 12).

TABLE 11
Amino acid sequences of antibodies and linkers
used for library construction

| | | | SEQ |
|---|---|---|---|
| | | | ID |
| Domain | Name | Amino Acid Sequence | NO: |
| VH | Elbow VH6 | ASTKGP | |
| Linker | | | |
| VH | Elbow VH7 | ASTKGPS | |
| Linker | | | |
| VH | Elbow VH8 | ASTKGPSV | |
| Linker | | | |
| VH | Elbow VH9 | ASTKGPSVF | |
| Linker | | | |
| VH | Elbow | ASTKGPSVFP | |
| Linker | VH10 | | |
| VH | Elbow | ASTKGPSVFPL | |
| Linker | VH11 | | |
| VH | Elbow | ASTKGPSVFPLA | |
| Linker | VH12 | | |
| VH | Elbow | ASTKGPSVFPLAP | |
| Linker | VH13 | | |
| VH | GS VH 6 | GGGGSG | |
| Linker | | | |
| VH | GS VH 7 | GGGGSGG | |
| Linker | | | |
| VH | GS VH 8 | GGGGSGGG | |
| Linker | | | |
| VH | GS VH 9 | GGGGSGGGG | |
| Linker | | | |
| VH | GS VH 10 | GGGGSGGGGS | |
| Linker | | | |
| VH | GS VH 11 | GGGGSGGGGSG | |
| Linker | | | |
| VH | GS VH 12 | GGGGSGGGGSGG | |
| Linker | | | |
| VH | GS VH 13 | GGGGSGGGGSGGG | |
| Linker | | | |
| VH | GS VH 14 | GGGGSGGGGSGGGG | |
| Linker | | | |
| VL | Elbow VL5 | TVAAP | |
| Linker | | | |
| VL | Elbow VL6 | TVAAPS | |
| Linker | | | |
| VL | Elbow VL7 | TVAAPSV | |
| Linker | | | |
| VL | Elbow VL8 | TVAAPSVF | |
| Linker | | | |
| VL | Elbow VL9 | TVAAPSVFI | |
| Linker | | | |
| VL | Elbow | TVAAPSVFIF | |
| Linker | VL10 | | |
| VL | Elbow | TVAAPSVFIFP | |
| Linker | VL11 | | |
| VL | Elbow | TVAAPSVFIFPP | |
| Linker | VL12 | | |
| VL | GS VL 5 | GGSGG | |
| Linker | | | |
| VL | GS VL 6 | GGSGGG | |
| Linker | | | |
| VL | GS VL 7 | GGSGGGG | |
| Linker | | | |
| VL | GS VL 8 | GGSGGGGS | |
| Linker | | | |
| VL | GS VL 9 | GGSGGGGSG | |
| Linker | | | |
| VL | GS VL 10 | GGSGGGGSGG | |
| Linker | | | |
| VL | GS VL 11 | GGSGGGGSGGG | |
| Linker | | | |
| VL | GS VL 12 | GGSGGGGSGGGG | |
| Linker | | | |
| VL | GS VL 13 | GGSGGGGSGGGGS | |
| Linker | | | |
| VH1 | 1B12.13 | EVQLQESGPGLVKPSETLSLTCTVSGFSLS | |
| | | DYGVSWIRQPPGKGLEWIGLIWGSGDTY | |
| | | YNSPLKSRLTISKDNSKSQVSLKLSSVTAA | |
| | | DTAVYYCAKQTNIWAYDLYSMDYWGQ | |
| | | GTLVTVSS | |
| VH1 | 1B12.21 | EVQLQESGPGLVKPSETLSLTCTVSGFSLS | |
| | | EFGVSWIRQPPGKGLEWIGLIWGGGDTY | |
| | | YNSPLKSRLTISKDNSKSQVSLKLSSVTAA | |
| | | DTAVYYCAKQRNLWAYDLYGMDYWGQ | |
| | | GTLVTVSS | |
| VH1 | 1B12.34 | EVQLQESGPGLVKPSETLSLTCTVSGFSLS | |
| | | DYGVSWIRQPPGKGLEWIGLIWGSGDTY | |
| | | YNSPLKSRLTISKDTSKSQVSLKLSSVTAA | |
| | | DTAVYYCAKQTNLWAYDLYSMDYWGQ | |
| | | GTLVTVSS | |
| VH1 | 1B12.A1 | EVQLQESGPGLVKPSETLSLTCTVSGFSLR | |
| | | DYGVSWIRQPPGKGLEWLGLIWGSGDTY | |
| | | YNSPLKSRLTISKDTSKSQVSLKLSSVTAA | |
| | | DTAVYYCAKQTNIWGYDLYGMDYWGQ | |
| | | GTLVTVSS | |
| VH1 | 1B12.A3 | EVQLQESGPGLVKPSETLSLTCTVSGFSLS | |
| | | DYGVSWIRQPPGKGLEWIGLIWGGGDTY | |
| | | YNSPLKSRLTISKDNSKSQVSLKLSSVTAA | |
| | | DTAVYYCARQTNLWAYDLYSMDYWGQ | |
| | | GTLVTVSS | |
| VH1 | E26.13 | EVQLVESGGGVVQPGRSLRLSCSASGFIFS | |
| | | RYDMSWVRQAPGKGLEWVAYISHGGAG | |
| | | TYYPDSVKGRFTISRDNSKNTLFLQMDSL | |
| | | RPEDTGVYFCARGGVTKGYFDVWGQGT | |
| | | PVTVSS | |
| VH1 | E26.35 | EVQLVESGGGVVQPGRSLRLSCSASGFIFS | |
| | | RYDMSWVRQAPGKGLEWVAYISHGGAG | |
| | | TYYPDSVKGRFTISRDNSKNTLFLQMDSL | |
| | | RAEDTAVYYCARGGVYKGYFDVWGQGT | |
| | | PVTVSS | |
| VH2 | 10F7M11 | EVQLVQSGAEVKKPGSSVKVSCKASGYT | |
| | | FTDYEIHWVRQAPGQGLEWMGVNDPES | |
| | | GGTFYNQKFDGRVTLTADESTSTAYMEL | |
| | | SSLRSEDTAVYYCTRYSKWDSFDGMDY | |
| | | WGQGTTVTVSS | |
| VH2 | B6-17G | EVQLVQSGAEVKKPGSSVKVSCKASGGS | |
| | | FGGYGIGWVRQAPGQGLEWMGGITPFFG | |
| | | FADYAQKFQGRVTITADESTTTAYMELS | |
| | | GLTSDDTAVYYCARDPNEFWGGYYSTH | |
| | | DFDSWGQGTTVTVSS | |
| VH2 | B6-5G | EVQLVQSGAEVKKPGESVKISCKASGGSF | |
| | | RSYGISWVRQAPGQGLEWMGGITHFFGIT | |
| | | DYAQKFQGRVTITADESTTTAYMELSGLT | |
| | | SDDTAVYYCAREPNDFWGGYYDTHDFD | |
| | | SWGQGTTVTVSS | |
| VL1 | 1B12.13 | DIQMTQSPSSLSASVGDRVTITCQTSTDID | |
| | | DDLNWYQQKPGKAPKLLISLASTLRPGVP | |
| | | SRFSGSGSGTDFTFTISSLQPEDFATYYCL | |
| | | QSDRLPLTFGQGTKLEIKR | |
| VL1 | 1B12.21 | DIQMTQSPSSLSASVGDRVTITCQTSQDID | |
| | | MDLNWYQQKPGKAPKLLISQGSTLWPGV | |
| | | PSRFSGSGSGTDFTFTISSLQPEDFATYYC | |
| | | LQTDSFPLTFGQGTKLEIKR | |
| VL1 | 1B12.34 | DIQMTQSPSSLSASVGDRVTITCQASQDID | |
| | | DDLNWYQQKPGKAPKLLISLASILRPGVP | |
| | | SRFSGSGSGTDFTFTISSLQPEDFATYYCL | |
| | | QSDSFPLTFGQGTKLEIKR | |
| VL1 | 1B12.A1 | DIQMTQSPSSLSASVGDRVTITCQASQDID | |
| | | MDLNWYQQKPGKAPKLLISQANTLPPGV | |
| | | PSRFSGSGSGTDFTFTISSLQPEDFATYYC | |
| | | LQSDWLPLTFGQGTKLEIKR | |
| VL1 | 1B12.A3 | DIQMTQSPSSLSASVGDRVTITCQASTDID | |
| | | DDLNWYQQKPGKAPKLLISLGSTLRPGVP | |
| | | SRFSGSGSGTDFTFTISSLQPEDFATYYCL | |
| | | QSDRLPLTFGQGTKLEIKR | |
| VL1 | E26 | DIQMTQSPSSLSASVGDRVTITCRASGNIH | |
| | (13 & | NYLTWYQQTPGKAPKLLIYNAKTLADGV | |
| | 35) | PSRFSGSGSGTDYTFTISSLQPEDIATYYC | |
| | | QHFWSIPYTFGQGTKLEIKR | |
| VL2 | 10F7M11 | DIQMTQSPSSLSASVGDRVTITCRASSGIIS | |
| | | YIDWFQQKPGKAPKRLIYATFDLASGVPS | |
| | | RFSGSGSGTDYTLTISSLQPEDFATYYCRQ | |
| | | VGSYPETFGQGTKLEIKR | |
| VL2 | B6-17G | EIVLTQSPDFQSVTPKEKVTITCRASQDIG | |
| | | SELHWYQQKPDQPPKLLIKYASHSTSGVP | |
| | | SRFSGSGSGTDFTLTINGLEAEDAGTYYC | |
| | | HQTDSLPYTFGPGTKVDIKR | |
| VL2 | B6-5G | EIVLTQSPDFQSVTPKEKVTITCRASQNIG | |
| | | SELHWYQQKPDQSPKLLIKYASHSISGVP | |
| | | SRFSGSGSGTDFTLTINGLEAEDAATYYC | |
| | | HQSDTLPHTFGQGTKVDIKR | |

TABLE 12
Domain distribution after library construction

| Domain | Type | Predicted % | Actual % |
|---|---|---|---|
| VH1 | 1B12 lineage | 50 | 45 |
| | E26 lineage | 50 | 53 |
| VH linker | Elbow | 50 | 60 |
| | GS | 50 | 38 |
| VH2 | B6 lineage | 50 | 32 |
| | 10F7M11 | 50 | 64 |
| VL1 | 1B12 lineage | 50 | 47 |
| | E26 lineage | 50 | 52 |
| VL linker | Elbow | 50 | 25 |
| | GS | 50 | 73 |
| VL2 | B6 lineage | 50 | 33 |
| | 10F7M11 | 50 | 67 |

### Example 13

### Selection of IL1β/IL17 DVD-Fab Library by Flow Cytometry

Optimal selection conditions for library sorting were determined from scouting experiments to be 5 nM IL1β and 5 nM IL17. Multiple selection rounds were completed with increasing stringency (see Table). For all selections sort gates were chosen to take the best simultaneous binders to both IL1β and IL17. After each sort round library DNA was isolated from yeast cells, transformed into E coli, and colony PCR sequencing performed to analyze the sort output. Listed in Table 13 and

Table 14 are the output sequences from round 3. Library output clones are converted to full DVD-Ig format for characterization as described in Example 4.

TABLE 13
Labeling and sorting conditions for IL1β/IL17 DVD-Fab library

| Sort | Antigen | Incubation | Incubation | | % |
|---|---|---|---|---|---|
| Round | Concentration | Temperature | Time | | cells sorted |
| R1 | 5 nM IL1β, 5 nM IL17 | RT | 5 | minutes | 0.52% of total |
| R2 | 1 nM IL1β, 1 nM IL17 | RT | 1 | minute | 0.35% of total |
| R3 | 1 nM IL1β, 1 nM IL17 | Ice | 1 | minute | 0.17% of total |

TABLE 13
Round 3 output sequences for Heavy and Light chains

| | | | | SEQ |
|---|---|---|---|---|
| Heavy | Count | Different | | ID |
| chain | observed | LC pairs | Heavy chain sequence | NO: |
| E26.35 | 16 | 7 | EVQLVESGGGVVQPGRSLRLSCSASGFIFS | |
| EL10 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSASTKGPSVFPEVQLVQSGAEVKK | |
| | | | PGSSVKVSCKASGYTFTDYEIHWVRQAP | |
| | | | GQGLEWMGVNDPESGGTFYNQKFDGRV | |
| | | | TLTADESTSTAYMELSSLRSEDTAVYYCT | |
| | | | RYSKWDSFDGMDYWGQGTTVTVSS | |
| E26.35 | 8 | 7 | EVQLVESGGGVVQPGRSLRLSCSASGFIFS | |
| EL13 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSASTKGPSVFPLAPEVQLVQSGAE | |
| | | | VKKPGSSVKVSCKASGYTFTDYEIHWVR | |
| | | | QAPGQGLEWMGVNDPESGGTFYNQKFD | |
| | | | GRVTLTADESTSTAYMELSSLRSEDTAVY | |
| | | | YCTRYSKWDSFDGMDYWGQGTTVTVSS | |
| E26.35 | 7 | 4 | EVQLVESGGGVVQPGRSLRLSCSASGFIFS | |
| EL12 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSASTKGPSVFPLAEVQLVQSGAEV | |
| | | | KKPGSSVKVSCKASGYTFTDYEIHWVRQ | |
| | | | APGQGLEWMGVNDPESGGTFYNQKFDG | |
| | | | RVTLTADESTSTAYMELSSLRSEDTAVYY | |
| | | | CTRYSKWDSFDGMDYWGQGTTVTVSS | |
| E26.35 | 5 | 2 | EVQLVESGGGVVQPGRSLRLSCSASGFIFS | |
| EL6 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSASTKGPEVQLVQSGAEVKKPGSS | |
| | | | VKVSCKASGYTFTDYEIHWVRQAPGQGL | |
| | | | EWMGVNDPESGGTFYNQKFDGRVTLTA | |
| | | | DESTSTAYMELSSLRSEDTAVYYCTRYSK | |
| | | | WDSFDGMDYWGQGTTVTVSS | |
| E26.35 | 5 | 4 | EVQLVESGGGVVQPGRSLRLSCSASGFIFS | |
| EL7 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSASTKGPSEVQLVQSGAEVKKPGS | |
| | | | SVKVSCKASGYTFTDYEIHWVRQAPGQG | |
| | | | LEWMGVNDPESGGTFYNQKFDGRVTLT | |
| | | | ADESTSTAYMELSSLRSEDTAVYYCTRYS | |
| | | | KWDSFDGMDYWGQGTTVTVSS | |
| E26.35 | 5 | 3 | EVQLVESGGGVVQPGRSLRLSCSASGFIFS | |
| EL8 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSASTKGPSVEVQLVQSGAEVKKPG | |
| | | | SSVKVSCKASGYTFTDYEIHWVRQAPGQ | |
| | | | GLEWMGVNDPESGGTFYNQKFDGRVTL | |
| | | | TADESTSTAYMELSSLRSEDTAVYYCTRY | |
| | | | SKWDSFDGMDYWGQGTTVTVSS | |
| E26.13 | 3 | 3 | EVQLVESGGGVVQPGRSLRLSCSASGFIFS | |
| EL10 | | | RYDMSWVRQAPGKGLEWVAYISHGGAG | |
| 10F7M11 | | | TYYPDSVKGRFTISRDNSKNTLFLQMDSL | |
| | | | RPEDTGVYFCARGGVTKGYFDVWGQGT | |
| | | | PVTVSSASTKGPSVFPEVQLVQSGAEVKK | |
| | | | PGSSVKVSCKASGYTFTDYEIHWVRQAP | |
| | | | GQGLEWMGVNDPESGGTFYNQKFDGRV | |
| | | | TLTADESTSTAYMELSSLRSEDTAVYYCT | |
| | | | RYSKWDSFDGMDYWGQGTTVTVSS | |
| E26.35 | 3 | 2 | EVQLVESGGGVVQPGRSLRLSCSASGFIFS | |
| GS10 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSGGGGSGGGGSEVQLVQSGAEVK | |
| | | | KPGSSVKVSCKASGYTFTDYEIHWVRQA | |
| | | | PGQGLEWMGVNDPESGGTFYNQKFDGR | |
| | | | VTLTADESTSTAYMELSSLRSEDTAVYYC | |
| | | | TRYSKWDSFDGMDYWGQGTTVTVSS | |
| E26.13 | 2 | 2 | EVQLVESGGGVVQPGRSLRLSCSASGFIFS | |
| EL13 | | | RYDMSWVRQAPGKGLEWVAYISHGGAG | |
| 10F7M11 | | | TYYPDSVKGRFTISRDNSKNTLFLQMDSL | |
| | | | RPEDTGVYFCARGGVTKGYFDVWGQGT | |
| | | | PVTVSSASTKGPSVFPLAPEVQLVQSGAE | |
| | | | VKKPGSSVKVSCKASGYTFTDYEIHWVR | |
| | | | QAPGQGLEWMGVNDPESGGTFYNQKFD | |
| | | | GRVTLTADESTSTAYMELSSLRSEDTAVY | |
| | | | YCTRYSKWDSFDGMDYWGQGTTVTVSS | |
| E26.13 | 2 | 2 | EVQLVESGGGVVQPGRSLRLSCSASGFIFS | |
| EL6 | | | RYDMSWVRQAPGKGLEWVAYISHGGAG | |
| 10F7M11 | | | TYYPDSVKGRFTISRDNSKNTLFLQMDSL | |
| | | | RPEDTGVYFCARGGVTKGYFDVWGQGT | |
| | | | PVTVSSASTKGPEVQLVQSGAEVKKPGSS | |
| | | | VKVSCKASGYTFTDYEIHWVRQAPGQGL | |
| | | | EWMGVNDPESGGTFYNQKFDGRVTLTA | |
| | | | DESTSTAYMELSSLRSEDTAVYYCTRYSK | |
| | | | WDSFDGMDYWGQGTTVTVSS | |
| E26.13 | 2 | 2 | EVQLVESGGGVVQPGRSLRLSCSASGFIFS | |
| EL8 | | | RYDMSWVRQAPGKGLEWVAYISHGGAG | |
| 10F7M11 | | | TYYPDSVKGRFTISRDNSKNTLFLQMDSL | |
| | | | RPEDTGVYFCARGGVTKGYFDVWGQGT | |
| | | | PVTVSSASTKGPSVEVQLVQSGAEVKKPG | |
| | | | SSVKVSCKASGYTFTDYEIHWVRQAPGQ | |
| | | | GLEWMGVNDPESGGTFYNQKFDGRVTL | |
| | | | TADESTSTAYMELSSLRSEDTAVYYCTRY | |
| | | | SKWDSFDGMDYWGQGTTVTVSS | |
| E26.35 | 2 | 2 | EVQLVESGGGVVQPGRSLRLSCSASGFIFS | |
| EL11 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSASTKGPSVFPLEVQLVQSGAEVK | |
| | | | KPGSSVKVSCKASGYTFTDYEIHWVRQA | |
| | | | PGQGLEWMGVNDPESGGTFYNQKFDGR | |
| | | | VTLTADESTSTAYMELSSLRSEDTAVYYC | |
| | | | TRYSKWDSFDGMDYWGQGTTVTVSS | |
| E26.35 | 2 | 2 | EVQLVESGGGVVQPGRSLRLSCSASGFIFS | |
| EL9 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSASTKGPSVFEVQLVQSGAEVKKP | |
| | | | GSSVKVSCKASGYTFTDYEIHWVRQAPG | |
| | | | QGLEWMGVNDPESGGTFYNQKFDGRVT | |
| | | | LTADESTSTAYMELSSLRSEDTAVYYCTR | |
| | | | YSKWDSFDGMDYWGQGTTVTVSS | |
| 1B12 mix | 1 | 1 | EVQLQESGPGLVKPSETLSLTCTVSGFSLS | |
| EL13 | | | DYGVSWIRQPPGKGLEWLGLIWGSGDTY | |
| 10F7M11 | | | YNSPLKSRLTISKDTSKSQVSLKLSSVTAA | |
| | | | DTAVYYCAKQTNIWGYDLYGMDYWGQ | |
| | | | GTLVTVSSASTKGPSVFPLAPEVQLVQSG | |
| | | | AEVKKPGSSVKVSCKASGYTFTDYEIHW | |
| | | | VRQAPGQGLEWMGVNDPESGGTFYNQK | |
| | | | FDGRVTLTADESTSTAYMELSSLRSEDTA | |
| | | | VYYCTRYSKWDSFDGMDYWGQGTTVTV | |
| | | | SS | |
| 1B12 mix | 1 | 1 | EVQLQESGPGLVKPSETLSLTCTVSGFSLS | |
| GS13 | | | DYGVSWIRQPPGKGLEWLGLIWGSGDTY | |
| 10F7M11 | | | YNSPLKSRLTISKDTSKSQVSLKLSSVTAA | |
| | | | DTAVYYCAKQTNIWGYDLYGMDYWGQ | |
| | | | GTLVTVSSGGGGSGGGGSGGGEVQLVQS | |
| | | | GAEVKKPGSSVKVSCKASGYTFTDYEIH | |
| | | | WVRQAPGQGLEWMGVNDPESGGTFYNQ | |
| | | | KFDGRVTLTADESTSTAYMELSSLRSEDT | |
| | | | AVYYCTRYSKWDSFDGMDYWGQGTTVT | |
| | | | VSS | |
| 1B12.A1 | 1 | 1 | EVQLQESGPGLVKPSETLSLTCTVSGFSLR | |
| EL12 | | | DYGVSWIRQPPGKGLEWLGLIWGSGDTY | |
| 10F7M11 | | | YNSPLKSRLTISKDTSKSQVSLKLSSVTAA | |
| | | | DTAVYYCAKQTNIWGYDLYGMDYWGQ | |
| | | | GTLVTVSSASTKGPSVFPLAEVQLVQSGA | |
| | | | EVKKPGSSVKVSCKASGYTFTDYEIHWV | |
| | | | RQAPGQGLEWMGVNDPESGGTFYNQKF | |
| | | | DGRVTLTADESTSTAYMELSSLRSEDTAV | |
| | | | YYCTRYSKWDSFDGMDYWGQGTTVTVS | |
| | | | S | |
| 1B12.A1 | 1 | 1 | EVQLQESGPGLVKPSETLSLTCTVSGFSLR | |
| EL13 | | | DYGVSWIRQPPGKGLEWLGLIWGSGDTY | |
| 10F7M11 | | | YNSPLKSRLTISKDTSKSQVSLKLSSVTAA | |
| | | | DTAVYYCAKQTNIWGYDLYGMDYWGQ | |
| | | | GTLVTVSSASTKGPSVFPLAPEVQLVQSG | |
| | | | AEVKKPGSSVKVSCKASGYTFTDYEIHW | |
| | | | VRQAPGQGLEWMGVNDPESGGTFYNQK | |
| | | | FDGRVTLTADESTSTAYMELSSLRSEDTA | |
| | | | VYYCTRYSKWDSFDGMDYWGQGTTVTV | |
| | | | SS | |
| E26.35 | 1 | 1 | EVQLVESGGGVVQPGRSLRLSCSASGFIFS | |
| GS11 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSGGGGSGGGGSGEVQLVQSGAEV | |
| | | | KKPGSSVKVSCKASGYTFTDYEIHWVRQ | |
| | | | APGQGLEWMGVNDPESGGTFYNQKFDG | |
| | | | RVTLTADESTSTAYMELSSLRSEDTAVYY | |
| | | | CTRYSKWDSFDGMDYWGQGTTVTVSS | |
| E26.35 | 1 | 1 | EVQLVESGGGVVQPGRSLRLSCSASGFIFS | |
| GS14 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSGGGGSGGGGSGGGGEVQLVQSG | |
| | | | AEVKKPGSSVKVSCKASGYTFTDYEIHW | |
| | | | VRQAPGQGLEWMGVNDPESGGTFYNQK | |
| | | | FDGRVTLTADESTSTAYMELSSLRSEDTA | |
| | | | VYYCTRYSKWDSFDGMDYWGQGTTVTV | |
| | | | SS | |
| E26.35 | 1 | 1 | EVQLVESGGGVVQPGRSLRLSCSASGFIFS | |
| GS7 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSGGGGSGGEVQLVQSGAEVKKPG | |
| | | | SSVKVSCKASGYTFTDYEIHWVRQAPGQ | |
| | | | GLEWMGVNDPESGGTFYNQKFDGRVTL | |
| | | | TADESTSTAYMELSSLRSEDTAVYYCTRY | |
| | | | SKWDSFDGMDYWGQGTTVTVSS | |
| E26.35 | 1 | 1 | EVQLVESGGGVVQPGRSLRLSCSASGFIFS | |
| GS8 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSGGGGSGGGEVQLVQSGAEVKKP | |
| | | | GSSVKVSCKASGYTFTDYEIHWVRQAPG | |
| | | | QGLEWMGVNDPESGGTFYNQKFDGRVT | |
| | | | LTADESTSTAYMELSSLRSEDTAVYYCTR | |
| | | | YSKWDSFDGMDYWGQGTTVTVSS | |
| E26.35 | 1 | 1 | EVQLVESGGGVVQPGRSLRLSCSASGFIFS | |
| GS9 | | | RYDMSWVRQAPGKGLEWVAYISHGGAG | |
| 10F7M11 | | | TYYPDSVKGRFTISRDNSKNTLFLQMDSL | |
| | | | RAEDTAVYYCARGGVYKGYFDVWGQGT | |
| | | | PVTVSSGGGGSGGGGEVQLVQSGAEVKK | |
| | | | PGSSVKVSCKASGYTFTDYEIHWVRQAP | |
| | | | GQGLEWMGVNDPESGGTFYNQKFDGRV | |
| | | | TLTADESTSTAYMELSSLRSEDTAVYYCT | |
| | | | RYSKWDSFDGMDYWGQGTTVTVSS | |
| Light | Count | Different | | |
| chain | observed | HC pairs | Light chain sequence | |
| E26 | 22 | 12 | DIQMTQSPSSLSASVGDRVTITCRASGNIH | |
| GS12 | | | NYLTWYQQTPGKAPKLLIYNAKTLADGV | |
| 10F7M11 | | | PSRFSGSGSGTDYTFTISSLQPEDIATYYC | |
| | | | QHFWSIPYTFGQGTKLEIKRGGSGGGGSG | |
| | | | GGGDIQMTQSPSSLSASVGDRVTITCRAS | |
| | | | SGIISYIDWFQQKPGKAPKRLIYATFDLAS | |
| | | | GVPSRFSGSGSGTDYTLTISSLQPEDFATY | |
| | | | YCRQVGSYPETFGQGTKLEIKR | |
| E26 | 16 | 8 | DIQMTQSPSSLSASVGDRVTITCRASGNIH | |
| GS13 | | | NYLTWYQQTPGKAPKLLIYNAKTLADGV | |
| 10F7M11 | | | PSRFSGSGSGTDYTFTISSLQPEDIATYYC | |
| | | | QHFWSIPYTFGQGTKLEIKRGGSGGGGSG | |
| | | | GGGSDIQMTQSPSSLSASVGDRVTITCRA | |
| | | | SSGIISYIDWFQQKPGKAPKRLIYATFDLA | |
| | | | SGVPSRFSGSGSGTDYTLTISSLQPEDFAT | |
| | | | YYCRQVGSYPETFGQGTKLEIKR | |
| E26 GS9 | 9 | 8 | DIQMTQSPSSLSASVGDRVTITCRASGNIH | |
| 10F7M11 | | | NYLTWYQQTPGKAPKLLIYNAKTLADGV | |
| | | | PSRFSGSGSGTDYTFTISSLQPEDIATYYC | |
| | | | QHFWSIPYTFGQGTKLEIKRGGSGGGGSG | |
| | | | DIQMTQSPSSLSASVGDRVTITCRASSGIIS | |
| | | | YIDWFQQKPGKAPKRLIYATFDLASGVPS | |
| | | | RFSGSGSGTDYTLTISSLQPEDFATYYCRQ | |
| | | | VGSYPETFGQGTKLEIKR | |
| E26 | 7 | 5 | DIQMTQSPSSLSASVGDRVTITCRASGNIH | |
| GS10 | | | NYLTWYQQTPGKAPKLLIYNAKTLADGV | |
| 10F7M11 | | | PSRFSGSGSGTDYTFTISSLQPEDIATYYC | |
| | | | QHFWSIPYTFGQGTKLEIKRGGSGGGGSG | |
| | | | GDIQMTQSPSSLSASVGDRVTITCRASSGII | |
| | | | SYIDWFQQKPGKAPKRLIYATFDLASGVP | |
| | | | SRFSGSGSGTDYTLTISSLQPEDAFTYYCR | |
| | | | QVGSYPETFGQGTLKEIKR | |
| E26 | 6 | 4 | DIQMTQSPSSLSASVGDRVTITCRASGNIH | |
| GS11 | | | NYLTWYQQTPGKAPKLLIYNAKTLADGV | |
| 10F7M11 | | | PSRFSGSGSGTDYTFTISSLQPEDIATYYC | |
| | | | QHFWSIPYTFGQGTKLEIKRGGSGGGGSG | |
| | | | GGDIQMTQSPSSLSASVGDRVTITCRASS | |
| | | | GIISYIDWFQQKPGKAPKRLIYATFDLASG | |
| | | | VPSRFSGSGSGTDYTLTISSLQPEDFATYY | |
| | | | CRQVGSYPETFGQGTKLEIKR | |
| E26 EL7 | 5 | 2 | DIQMTQSPSSLSASVGDRVTITCRASGNIH | |
| 10F7M11 | | | NYLTWYQQTPGKAPKLLIYNAKTLADGV | |
| | | | PSRFSGSGSGTDYTFTISSLQPEDIATYYC | |
| | | | QHFWSIPYTFGQGTKLEIKRTVAAPSVDI | |
| | | | QMTQSPSSLSASVGDRVTITCRASSGIISYI | |
| | | | DWFQQKPGKAPKRLIYATFDLASGVPSRF | |
| | | | SGSGSGTDYTLTISSLQPEDFATYYCRQV | |
| | | | GSYPETFGQGTKLEIKR | |
| E26 GS8 | 4 | 2 | DIQMTQSPSSLSASVGDRVTITCRASGNIH | |
| 10F7M11 | | | NYLTWYQQTPGKAPKLLIYNAKTLADGV | |
| | | | PSRFSGSGSGTDYTFTISSLQPEDIATYYC | |
| | | | QHFWSIPYTFGQGTKLEIKRGGSGGGGSD | |
| | | | IQMTQSPSSLSASVGDRVTITCRASSGIISY | |
| | | | IDWFQQKPGKAPKRLIYATFDLASGVPSR | |
| | | | FSGSGSGTDYTLTISSLQPEDFATYYCRQV | |
| | | | GSYPETFGQGTKLEIKR | |
| E26 GS6 | 3 | 2 | DIQMTQSPSSLSASVGDRVTITCRASGNIH | |
| 10F7M11 | | | NYLTWYQQTPGKAPKLLIYNAKTLADGV | |
| | | | PSRFSGSGSGTDYTFTISSLQPEDIATYYC | |
| | | | QHFWSIPYTFGQGTKLEIKRGGSGGGGDIQ | |
| | | | MTQSPSSLSASVGDRVTITCRASSGIISYID | |
| | | | WFQQKPGKAPKRLIYATFDLASGVPSRFS | |
| | | | GSGSGTDYTLTISSLQPEDFATYYCRQVG | |
| | | | SYPETFGQGTKLEIKR | |
| 1B12.A1 | 2 | 2 | DIQMTQSPSSLSASVGDRVTITCQASQDID | |
| EL8 | | | MDLNWYQQKPGKAPKLLISQANTLPPGV | |
| 10F7M11 | | | PSRFSGSGSGTDFTFTISSLQPEDFATYYC | |
| | | | LQSDWLPLTFGQGTKLEIKRTVAAPSVFD | |
| | | | IQMTQSPSSLSASVGDRVTITCRASSGIISY | |
| | | | IDWFQQKPGKAPKRLIYATFDLASGVPSR | |
| | | | FSGSGSGTDYTLTISSLQPEDFATYYCRQV | |
| | | | GSYPETFGQGTKLEIKR | |
| E26 GS5 | 2 | 1 | DIQMTQSPSSLSASVGDRVTITCRASGNIH | |
| 10F7M11 | | | NYLTWYQQTPGKAPKLLIYNAKTLADGV | |
| | | | PSRFSGSGSGTDYTFTISSLQPEDIATYYC | |
| | | | QHFWSIPYTFGQGTKLEIKRGGSGGDIQM | |
| | | | TQSPSSLSASVGDRVTITCRASSGIISYIDW | |
| | | | FQQKPGKAPKRLIYATFDLASGVPSRFSG | |
| | | | SGSGTDYTLTISSLQPEDFATYYCRQVGS | |
| | | | YPETFGQGTKLEIKR | |
| 1B12.A1 | 1 | 1 | DIQMTQSPSSLSASVGDRVTITCQASQDID | |
| GS7 | | | MDLNWYQQKPGKAPKLLISQANTLPPGV | |
| | | | PSRFSGSGSGTDFTFTISSLQPEDFATYYC | |
| | | | LQSDWLPLTFGQGTLKEIKRGGSGGGGDI | |
| | | | QMTQSPSSLSASVGDRVTITCRASSGIISYI | |
| | | | DWFQQKPGKAPKRLIYATFDLASGVPSRF | |
| | | | SGSGSGTDYTLTISSLQPEDFATYYCRQV | |
| | | | GSYPETFGQGTKLEIKR | |
| 1B12 | 1 | 1 | DIQMTQSPSSLSASVGDRVTITCQASQDID | |
| GS10 | | | MDMNWYQQKPGKAPKLLISQANTLPPG | |
| 10F7M11 | | | VHSRFSGSGSGTDFTFTISSLQPEDFATYY | |
| | | | CLQSDWLPLTFGQGTKLEIKRGGSGGGGS | |
| | | | GGDIQMTQSPSSLSASVGDRVTITCRASS | |
| | | | GIISYIDWFQQKPGKAPKRLIYATFDLASG | |
| | | | VPSRFSGSGSGTDYTLTISSLQPEDFATYY | |
| | | | CRQVGSYPETFGQGTKLEIKR | |
| E26 EL12 | 1 | 1 | DIQMTQSPSSLSASVGDRVTITCRASGNIH | |
| 10F7M11 | | | NYLTWYQQTPGKAPKLLIYNAKTLADGV | |
| | | | PSRFSGSGSGTDYTFTISSLQPEDIATYYC | |
| | | | QHFWSIPYTFGQGTKLEIKRTVAAPSVFIF | |
| | | | PPDIQMTQSPSSLSASVGDRVTITCRASSG | |
| | | | IISYIDWFQQKPGKAPKRLIYATFDLASGV | |
| | | | PSRFSGSGSGTDYTLTISSLQPEDFATYYC | |
| | | | RQVGSYPETFGQGTKLEIKR | |
| E26 EL5 | 1 | 1 | DIQMTQSPSSLSASVGDRVTITCRASGNIH | |
| 10F7M11 | | | NYLTWYQQTPGKAPKLLIYNAKTLADGV | |
| | | | PSRFSGSGSGTDYTFTISSLQPEDIATYYC | |
| | | | QHFWSIPYTFGQGTKLEIKRTVAAPDIQM | |
| | | | TQSPSSLSASVGDRVTITCRASSGIISYIDW | |
| | | | FQQKPGKAPKRLIYATFDLASGVPSRFSG | |
| | | | SGSGTDYTLTISSLQPEDFATYYCRQVGS | |
| | | | YPETFGQGTKLEIKR | |
| E26 EL6 | 1 | 1 | DIQMTQSPSSLSASVGDRVTITCRASGNIH | |
| 10F7M11 | | | NYLTWYQQTPGKAPKLLIYNAKTLADGV | |
| | | | PSRFSGSGSGTDYTFTISSLQPEDIATYYC | |
| | | | QHFWSIPYTFGQGTKLEIKRTVAAPSDIQ | |
| | | | MTQSPSSLSASVGDRVTITCRASSGIISYID | |
| | | | WFQQKPGKAPKRLIYATFDLASGVPSRFS | |
| | | | GSGSGTDYTLTISSLQPEDFATYYCRQVG | |
| | | | SYPETFGQGTKLEIKR | |

TABLE 14
Round 3 output sequences for DVDs

| DVD | Count Observed |
|---|---|
| E26.35+ 10F7M11, EL10, GS12 | 6 |
| E26.35+ 10F7M11, EL10, EL7 | 3 |
| E26.35+ 10F7M11, EL10, GS11 | 3 |
| E26.35+ 10F7M11, EL6, GS13 | 3 |
| E26.35+ 10F7M11, EL8, GS12 | 3 |
| E26.35+ 10F7M11, EL12, GS12 | 2 |
| E26.35+ 10F7M11, EL12, GS13 | 2 |
| E26.35+ 10F7M11, EL12, GS6 | 2 |
| E26.35 + 10F7M11, EL13, GS10 | 2 |
| E26.35 + 10F7M11, GS10, GS13 | 2 |
| 1B12 mix + 10F7M11, EL13, GS10 | 1 |
| 1B12 mix + 10F7M11, GS13, EL8 | 1 |
| 1B12.A1 + 10F7M11, EL12, EL8 | 1 |
| 1B12.A1 + 10F7M11, EL13, GS7 | 1 |
| E26.13 + 10F7M11, EL10, GS10 | 1 |
| E26.13 + 10F7M11, EL10, GS12 | 1 |
| E26.13 + 10F7M11, EL10, GS9 | 1 |
| E26.13 + 10F7M11, EL13, GS11 | 1 |
| E26.13 + 10F7M11, EL13, GS5 | 1 |
| E26.13 + 10F7M11, EL6, GS10 | 1 |
| E26.13 + 10F7M11, EL6, GS12 | 1 |
| E26.13 + 10F7M11, EL8, GS12 | 1 |
| E26.13 + 10F7M11, EL8, GS9 | 1 |
| E26.35 + 10F7M11, EL10, GS10 | 1 |
| E26.35 + 10F7M11, EL10, GS13 | 1 |
| E26.35 + 10F7M11, EL10, GS6 | 1 |
| E26.35 + 10F7M11, EL10, GS9 | 1 |
| E26.35 + 10F7M11, EL11, GS12 | 1 |
| E26.35 + 10F7M11, EL11, GS9 | 1 |
| E26.35 + 10F7M11, EL12, EL5 | 1 |
| E26.35 + 10F7M11, EL13, EL12 | 1 |
| E26.35 + 10F7M11, EL13, EL6 | 1 |
| E26.35 + 10F7M11, EL13, GS12 | 1 |
| E26.35 + 10F7M11, EL13, GS13 | 1 |
| E26.35 + 10F7M11, EL13, GS8 | 1 |
| E26.35 + 10F7M11, EL13, GS9 | 1 |
| E26.35 + 10F7M11, EL6, GS12 | 1 |
| E26.35 + 10F7M11, EL7, GS11 | 1 |
| E26.35 + 10F7M11, EL7, GS12 | 1 |
| E26.35 + 10F7M11, EL7, GS13 | 1 |
| E26.35 + 10F7M11, EL7, GS9 | 1 |
| E26.35 + 10F7M11, EL8, EL7 | 1 |
| E26.35 + 10F7M11, EL8, GS13 | 1 |
| E26.35 + 10F7M11, EL9, GS11 | 1 |
| E26.35 + 10F7M11, EL9, GS9 | 1 |
| E26.35 + 10F7M11, GS10, GS12 | 1 |
| E26.35 + 10F7M11, GS11, GS8 | 1 |
| E26.35 + 10F7M11, GS14, GS10 | 1 |
| E26.35 + 10F7M11, GS7, GS12 | 1 |
| E26.35 + 10F7M11, GS8, GS9 | 1 |
| E26.35 + 10F7M11, GS9, GS13 | 1 |

### Example 14

### Construction of Full-Length DVD-Ig for Yeast Display

A DLL4/VEGF DVD (comprising the VH and VL domains of an anti-DLL4 antibody and an anti-VEGF antibody) was cloned into the pFabB yeast expression vector as both a DVD-Fab and full length DVD-Ig. Briefly, the VL coding region of the DVD was amplified and combined by overlapping PCR with a portion of the pFabB vector and the DVD heavy chain (either the VH region or the full VH+Fc), excluding stop codon. For the full length DVD another portion of the pFab vector was also included in the overlapping PCR for cloning purposes. For the DVD-Fab construct pFabB was linearized with BsiWI, BamHI, and SalI; for the DVD-Ig the pFabB was linearized with BsiWI, BamHI, and Pad and PCR products were inserted by homologous recombination. After sequence confirmation, the DVD-Fab and DVD-Ig yeast display vectors were transformed into chemically competent S. cerevisiae cells.

### Example 15

### Flow Cytometric Analysis of Full-Length DVD-Ig Yeast Cells

Yeast cells were induced for protein expression followed by flow cytometry staining experiments to verify display and antigen binding. Display of either DVD-Fab or DVD-Ig heavy chain was monitored by staining for a V5 tag, light chain was monitored by use of an anti-hCK reagent, and the presence of the full-length DVD-Ig was monitored by a polyclonal anti-hFc reagent. Table 5 lists the percent of cells showing display of heavy chain and light chain using the various staining reagents. Note that only the full-length DVD-Ig shows reactivity with the anti-hFc reagent. Simultaneous antigen binding to both VEGF (visualized using biotinylated VEGF and streptavidin-PE) and DLL4 (Alexa647 conjugated DLL4) was observed for both DVD-Fab and DVD-Ig. Table 6 shows the mean fluorescence intensity (MFI) for antigen binding of anti-V5 positive cells.

TABLE 5
Yeast cells binding to heavy chain and light chain reagents

| | % α-V5+ cells | % α-hFc+ cells | % α-hCK+ cells |
|---|---|---|---|
| DVD-Fab | 64 | 0 | 54 |
| DVD-Ig | 63 | 51 | 24 |

TABLE 6
Anti-V5 positive yeast cells simultaneous binding to VEGF and DLL4

| | No Ag MFI | VEGF MFI | No Ag MFI | DLL4 MFI |
|---|---|---|---|---|
| | (PE) | (PE) | (Alexa 647) | (Alexa 647) |
| DVD-Fab | 168 | 493 | 30 | 333 |
| DVD-Ig | 194 | 333 | 35 | 105 |

1. A diverse library of binding proteins comprising a first polypeptide chain having the general formula VH1-(X1)n-VH2-C—(X2)n, wherein VH1 is a first heavy chain variable domain, X1 is a linker with the proviso that it is not a constant domain, VH2 is a second heavy chain variable domain, C is a heavy chain constant domain, X2 is a cell surface protein, and n is 0 or 1, and wherein the amino acid sequences of VH1, VH2 and/or X1 independently vary within the library.

2. The library of claim 2, wherein the binding proteins further comprise a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional antigen binding sites.

3. The diverse library of claim 2, wherein the first and second polypeptide chains combine to form a DVD-Fab or a full length DVD-Ig.

4. The library of claim 2, wherein the amino acid sequences of VL1, VL2 and/or Y1 independently vary within the library.

5. The library of claim 1, wherein the amino acid sequences of at least one CDR of VH1, VH2, VL1 or VL2 independently varies within the library.

6. The library of claim 1, wherein the amino acid sequences of HCDR3 of VH1, VH2 independently vary within the library.

7. The library of claim 1, wherein the amino acid sequences of HCDR1 and HCDR2 of VH1 or VH2 independently vary within the library.

8. The library of claim 1, wherein the amino acid sequences of HCDR1, HCDR2 and HCDR3 of VH1 or VH2 independently vary within the library.

9. The library of claim 1, wherein the amino acid sequences of HCDR3 of VL1 or VL2 independently vary within the library.

10. The library of claim 1, wherein the amino acid sequences of HCDR1 and HCDR2 of VL1 or VL2 independently vary within the library.

11. The library of claim 1, wherein the amino acid sequences of HCDR1, HCDR2 and HCDR3 of VL1 or VL2 independently vary within the library.

12. The library of claim 1, wherein X1 independently varies within the library and wherein X1 is selected from the amino acid sequences set forth in Table 7 and/or 11.

13. The library of claim 1, wherein Y1 independently varies within the library and wherein Y1 is selected from the amino acid sequences set forth in Table 7 and/or 11.

14. The library of claim 1, wherein X2 comprises the Aga2p polypeptide.

15. The library of claim 1, wherein the library of binding proteins share at least 70, 75, 80, 85, 90, 95, 96, 97, 98, or 99 amino acid sequence identity with a reference binding protein.

16. The library of claim 1, wherein VH1 and VH2 of the reference binding protein specifically bind to different antigens.

17. A diverse library of polynucleotides encoding the first and/or second polypeptide chains of the diverse library of binding proteins of claim 1.

18. A diverse library of expression vectors comprising the diverse library of polynucleotides of claim 17.

19. A library of transformed host cells, expressing the diverse library of binding proteins of claim 1.

20. The library of transformed host cells of claim 19, wherein the binding proteins are anchored on the cell surface.

21. The library of transformed host cells of claim 19, wherein the binding proteins are anchored on the cell surface through Aga1p.

22. The library of transformed host cells of claim 19, wherein the host cells are eukaryotic.

23. The library of transformed host cells of claim 22, wherein the host cells are yeast.

24. The library of transformed host cells of claim 23, wherein the yeast is selected from the group consisting of Saccharomyces cerevisiae, Saccharomyces carlsbergensis, Candida albicans, Candida kefyr, Candida tropicalis, Cryptococcus laurentii, Cryptococcus neoformans, Hansenula anomala, Hansenula polymorpha, Kluyveromyces fragilis, Kluyveromyces lactis, Kluyveromyces marxianus, Pichia pastoris, Rhodotorula rubra, Schizosaccharomyces pombe and Yarrowia lipolytica.

25. The library of transformed host cells of claim 23, wherein the yeast is Saccharomyces cerevisiae.

26. A method of selecting a binding protein that specifically binds to a target antigen, the method comprising:
a) providing a diverse library of transformed host cells expressing a diverse library of binding proteins of claim 1;
b) contacting the host cells with the target antigen; and
c) selecting a host cell that bind to the target antigen, thereby identifying a binding protein that specifically binds to a target antigen.

27. A method of selecting a binding protein that specifically binds to a first and a second target antigen simultaneously, the method comprising:
a) providing a diverse library of transformed host cells expressing the diverse library of binding proteins, wherein the diverse library of binding proteins comprises a first polypeptide chain having the general formula VH1-(X1)n-VH2-C—(X2)n, wherein VH1 is a first heavy chain variable domain, X1 is a linker with the proviso that it is not a constant domain, VH2 is a second heavy chain variable domain, C is a heavy chain constant domain, X2 is a cell surface protein, and n is 0 or 1, and wherein the amino acid sequences of VH1, VH2 and/or X1 independently vary within the library;
b) contacting the host cells with the first and second target antigen; and
c) selecting a host cell that bind to the first and second target antigen, thereby identifying a binding protein that specifically binds to a first and a second target antigen simultaneously.

28. The method of claim 26, wherein host cells that bind to the first and/or second antigen are selected by Magnetic Activated Cell Sorting using magnetically labeled antigen.

29. The method of claim 26, wherein host cells that bind to the first and/or second antigen are selected by Fluorescence Activated Cell Sorting using fluorescently labeled antigen.

30. The method of claim 26, further comprising isolating the binding protein-encoding polynucleotide sequences from the host cells selected in step (c).

31. A method of producing a binding protein, comprising expressing in a host cell a binding protein that was selected using the method of claim 26.

32. A multivalent binding protein having the general formula VH1-(X1)n-VH2-C—X2, wherein VH1 is a first heavy chain variable domain, X1 is a linker with the proviso that it is not a constant domain, VH2 is a second heavy chain variable domain, C is a heavy chain constant domain, X2 is an anchoring moiety, and n is 0 or 1.

33. The multivalent binding protein of claim 32 further comprising a second polypeptide chain having the general formula VL1-(Y1)n-VL2-C, wherein VL1 is a first light chain variable domain, Y1 is a linker with the proviso that it is not a constant domain, VL2 is a second light chain variable domain, C is a light chain constant domain, n is 0 or 1, wherein the VH1 and VH2 of the first polypeptide chain and VL1 and VL2 of second polypeptide chains of the binding protein combine form two functional antigen binding sites.

34. The binding protein of claim 33 which is a DVD-Fab molecule or a full length DVD-Ig.

35. The binding protein of claim 33, wherein the anchoring moiety cell surface protein.

36. The binding protein of claim 33, wherein the anchoring moiety comprises the Aga2p polypeptide.

37. A polynucleotide encoding a binding protein of claim 32.

38. A host cell expressing a binding protein of claim 32.
